(19) United States
(12) Patent Application Publication
(10) Pub. No.: US 2004/0170602 A1
(43) Pub. Date: Sep. 2, 2004

(54) Dominant negative proteins and methods thereof

(76) Inventors: John R. Desjarlais, Pasadena, CA (US); Malu Lourdes G. Tansey, Coppell, TX (US); Bassil I. Dahiyat, Altadena, CA (US)

Correspondence Address: DORSEY & WHITNEY LLP, INTELLECTUAL PROPERTY DEPARTMENT, 4 EMBARCADERO CENTER, SUITE 3400, SAN FRANCISCO, CA, 94111, US

(21) Appl. No.: 10/611,399

(22) Filed: Jul. 1, 2003

Related U.S. Application Data
(63) Continuation-in-part of application No. 10/338,083, filed on Jan. 6, 2003.
(60) Provisional application No. 60/345,805, filed on Jan. 4, 2002.
(60) Provisional application No. 60/373,453, filed on Apr. 17, 2002.

(51) Int. Cl. A61K 38/19

(52) U.S. Cl. 424/85.1

(57) ABSTRACT

The invention relates to novel proteins with TNFSF antagonist activity and nucleic acids encoding these proteins. The invention further relates to the use of the novel proteins in the treatment of TNFSF-related disorders.

This application claims the benefit of the filing date of U.S. Ser. No. 60/345,805, filed Jan. 4, 2002, U.S. Ser. No. 60/373,453, filed Apr. 17, 2002, U.S. Ser. No. 10/338083, and PCT 03/00394, both filed Jan. 6, 2003, all of which are expressly incorporated by reference in their entirety.

### FIELD OF THE INVENTION

The invention relates to novel proteins of the Tumor Necrosis Factor Super Family (TNFSF) with antagonist activity and nucleic acids encoding these proteins. The invention further relates to the use of the novel proteins in the treatment of TNFSF related disorders, such as autoimmune conditions, including but not limited to rheumatoid arthritis, sepsis and Crohn's disease, as well as peripheral nerve injury and demyelinating disorders.

### BACKGROUND OF THE INVENTION

Mullticellular organisms consist of an intricate and ordered society of individual cells that must communicate to maintain and regulate their functions. This is achieved through a complex and highly regulated network of hormones, chemica mediators, chemokines and other cytokines, acting as ligand for intra- or extracellular receptors. (Bodmer, J-L., et al., (2002) TIBS, 27, 19-26. Such ligands are often activated by ligand-induced oligomerization or conformational changes (Heldin, C-H., (1995) Cell, 80, 213-223). Designing proteins that interfere with intracellular signaling processes is of considerable interest, as many of these proteins would be useful in the treatment of anemia, cancer, diabetes, inflammation, neurological and growth disorders and other disease states.

The TNFSF proteins constitute an important class of cytokines that participate in a variety of cellular and intracellular signaling processes. The prototype of the family, Tumor Necrosis Factor Alpha (TNFA ), originally discovered for its in vivo effect causing tumors to regress, is a key mediator of inflammation. The TNFSF currently constitutes at least 18 unique cytokines that exist in secreted and membrane-bound forms. These proteins are important regulators of innate and adaptive immune responses and developmental events. They are synthesized as type 2 membrane proteins and fold into conserved β-pleated sheet structures that trimerize. While most TNFSF members form homotrimers, a few exceptions exist. While lymphotoxin a, but not β, can form homotrimers, the two can also form active heterotrimers with each other. Similarly, APRIL and BLyS also form both homotrimers and heterotrimers together. Many of the TNF family members remain membrane-bound and serve as cell contact mediated regulators, while others are cleaved from the membrane to release the extracellular domain as a regulator.

The receptors for TNF family members also represent a family of structurally related molecules, including at least 26 receptors and/or receptor decoy molecules. The extracellular domains of members of this family are composed of multiple repeats of a cysteine-rich domain (CRD), a small protein domain containing six conserved cysteines that form three disulfide bonds. The intracellular domains of these receptors are more diverse, although many members of the family contain a death domain that mediates apoptosis and other receptor signaling events. These members are all capable of inducing apoptosis via interaction with one or more intracellular adaptor molecules that also contain death domains. Other signaling receptors of this family signal via interactions with a family of adaptor molecules called TRAFs (TNF receptor associated factors). Signaling through TNFSF receptors is triggered by binding of an oligomeric (and for the most part, trimeric) TNFSF ligand. Many members of the TNFSF ligand family interact and signal through more than one receptor (e.g. TNFA interacts with receptors TNFR1 and TNFR2). In addition, more than one member of the TNFSF ligand family can interact with one receptor (e.g. TNFA and TNFB both interact with TNFR1 and TNFR2). In most known cases, TNFSF receptors are activating receptors. However, in other cases, some TNFSF receptors are inhibitory. In the BLyS family of receptors, BAFF-R is an activating receptor that promotes B-cell survival and maturation, while there is growing evidence that TACI is an inhibitory receptor that prevents B-cell survival.

The three-dimensional structures of TNFSF members are very similar, made up of a sandwich of two anti-parallel beta-sheets with the “jelly roll” or Greek key topology. In addition, all characterized members of the family assemble into trimeric complexes. The cognate receptors of the TNF family ligands make up a related superfamily of receptors. Furthermore, there appears to be significant conservation of the mode of receptor binding. In general, each receptor monomer binds within the cleft formed between two of the ligand monomers. The overall similarity in tertiary and quaternary structures of both the ligands and their complexes with receptors suggests that well-proven strategies for inhibition of one ligand-receptor system may be transferable to the other proteins in the family. However, a mechanism for extension to other members of the family has not previously been defined. Thus the present invention provides methods for the creation of variants of each member of the TNFSF that are antagonistic.

A need still exists for proteins that can interfere with intracellular signaling processes. Thus, it is an object of the present invention to provide proteins comprising multiple TNF superfamily receptor-interaction domains that are modified such that each domain has significantly reduced affinity and/or signaling capacity for the cognate receptor(s). Such linked domains preferably retain association with individual monomer domains, but exhibit a dominant-negative phenotype, antagonizing the action of related naturally occurring domains via their sequestration into inactive oligomeric complexes.

### SUMMARY OF THE INVENTION

In accordance with the objects outlined above, the present invention provides variants of extracellular domains of TNFSF proteins that antagonize naturally occurring TNFSF proteins.

The present invention provides variant TNFSF proteins comprising an amino acid sequence that have at least one modification as compared to the a naturally occurring TNFSF protein sequence, wherein said variant TNFSF proteins will physically interact with a naturally occurring TNFSF proteins to form mixed oligomers substantially incapable of activating receptor signaling.

In another embodiment, variant TNFSF proteins of the invention are in at least monomeric form and comprise amino acid sequences that have at least one modification as compared to a naturally occurring TNFSF protein sequence, wherein said variant TNFSF proteins will interact with a receptor interface at at least one receptor binding site to render said receptor substantially incapable of activating receptor signaling.

Preferably, a variant TNFSF protein of the present invention has at least one receptor contact domain that has reduced affinity for a desired receptor as compared to its corresponding wild-type TNFSF protein and retains the ability to interact with other receptor interaction domains.

More specifically, variant TNFSF proteins of the present invention physically interact with a naturally occurring TNFSF protein to reduce the ability of the naturally occurring TNFSF to activate at least one receptor. Variant TNFSF proteins of the present invention may interact with its corresponding naturally occurring TNFSF protein or a non-corresponding naturally occurring TNFSF protein.

More specifically, variant TNFSF proteins comprising at least one modified receptor-contact domain that has reduced affinity and/or signaling capacity for a desired receptor wherein said protein cannot substantially activate the desired receptor, but retains the ability to interact with other TNFSF proteins.

In an additional aspect, the invention provides methods of antagonizing a naturally occurring TNFSF protein comprising contacting a naturally occurring TNFSF monomer protein with a variant TNFSF monomer protein comprising at least a variant extracellular domain of a TNFSF protein, to form a mixed TNFSF oligomer. In some cases, the mixed oligomers have reduced receptor signaling as compared to wild-type oligomers, for example when the mixed oligomer interacts with a receptor interface in at least one receptor binding site to render the receptor substantially incapable of activating receptor signaling. Alternatively or additionally, the mixed oligomers are substantially incapable of activating receptor signaling.

The present invention relates to the use of variants of TNF superfamily protein ligands for the inhibition or treatment of a variety of diseases. The variants are specifically engineered to remove or reduce their biological signaling capacity, while retaining their ability to function as oligomeric species, either in complex with themselves or with naturally occurring members of the superfamily.

In a preferred embodiment, variant TNFSF proteins are engineered to yield significantly reduced signaling through TNFSF receptors compared to wild-type TNFSF proteins while maintaining affinity for other TNFSF proteins to allow formation of mixed olimers, most preferably trimers. Such variant TNFSF proteins are referred to as “dominant negative TNFSF variants” or “DN-TNFSF”. The dominant negative TNFSF variants act by sequestering one or more naturally occurring TNFSF proteins in mixed heterotrimers that are substantially incapable of appreciably activating biological signaling. Consequently, DN-TNFSF proteins act to antagonize the action of naturally occurring TNFSF.

The present invention provides non-naturally occurring variant TNFSF proteins (e.g. proteins not found in nature) comprising amino acid sequences with at least one modification compared to the wild-type TNFSF proteins. Examples of suitable proteins include but are not limited to TNF-α, lymphotoxin-α, lymphotoxin-β, Fas ligand (FasL), TRAIL, CD40 ligand (CD40L), CD30 ligand, CD27 ligand, Ox40 ligand, APRIL, BLyS, 4-IBBL, TRANCE and RANKL (OPGL), and any other protein that is recognized to be a member of the TNFSF.

Preferred embodiments utilize variant TNFSF proteins that interact-with one or more wild-type TNFSF members to form mixed trimers incapable of substantially activating receptor signaling. Preferably, variant TNFSF proteins with at least one amino acid change are used as compared to a wild-type TNFSF protein.

In another preferred embodiment, variant TNFSF proteins with reduced binding and/or signaling through one receptor retain the ability to partially or fully interact with and signal through a different TNFSF receptor. This feature is particularly desirable for cases in which a TNFSF ligand protein naturally interacts with more than one receptor and the signaling through each receptor has different biological effects, such as inhibition versus activation.

In another preferred embodiment, modifications may be made either individually or in combination, with any combination being possible. Preferred embodiments utilize at least one, and preferably more, positions in each variant TNFSF protein. For example, amino acid substitutions may combined to form double variants or triple point variants.

In a further embodiment, a TNFSF molecule may be chemically modified, for example by PEGylation or glycosylation.

In another aspect, portions of the N- or C-termini may deleted. In a further embodiment, a TNFSF molecule may be circularly permuted.

In an additional aspect, the two or more receptor interaction domains of the variant proteins are covalently linked by a linker peptide or by other means. Preferably, the linker peptide is a sequence of at least one and not more than about 30 amino acid residues and comprises one or more of the following amino acid residues: Gly, Ser, Ala, or Thr.

In a further aspect, the invention provides recombinant nucleic acids encoding the non-naturally occurring variant TNFSF proteins, expression vectors, and host cells.

In an additional aspect, the invention provides methods of producing a non-naturally occurring variant TNFSF protein comprising culturing the host cell of the invention under conditions suitable for expression of the nucleic acid.

In a further aspect, the invention provides pharmaceutical compositions comprising a variant TNFSF protein of the invention and a pharmaceutical carrier.

In a further aspect, the invention provides methods for treating a TNFSF related disorder comprising administering a variant TNFSF protein of the invention to a patient.

### BRIEF DESCRIPTION OF THE DRAWINGS

FIG. 1 illustrates a general mechanism by which a dominant negative TNFSF protein may antagonize the action of a naturally occurring TNFSF protein. Ovals represent a TNFSF protein monomer and triangles represent receptor molecules. Naturally occurring variant TNFSF typically signal by organizing receptors into an active complex. Variant TNFSF proteins are modified (bumps and sticks) within their receptor contact domains such that receptor binding and/or signaling is reduced. When variant TNFSF trimers are incubated with naturally occurring TNFSF protein trimers, they equilibrate to form four different trimer species, three of which are inactive. At sufficient concentrations of dominant-negative variant TNFSF, essentially all of the naturally occurring TNFSF will be sequestered into inactive heterotrimeric complexes. Through this and related mechanisms of inactivating TNFSF proteins, the dominant-negative TNFSF variants will exert their therapeutic effects.

FIG. 2 depicts the structural superposition of TNFSF Ligand Monomers. Experimentally determined structures of CD40L (1ALY), RANKL (1JTZ), TNFB (1TNR), and TRAIL (1DG6) are shown superimposed onto the structure of TNFA (1TNF).

FIG. 3 shows a Multiple Sequence Alignment (MSA) of human TNFSF members. Amino acids in the alignment are differentially highlighted according to 6 groupings of physicochemical properties as follows: nonpolar (A, C, I, L, M, V), aromatic (F, W, Y), neutral polar (N, Q, S, T), charged positive (H, K, R), charged Negative (D, E), and conformational (G, P). FIG. 3 also shows position numberings of each individual sequence. For TNF-α (TNFA) and TNFB (LT-α), the numbering is based on current convention. For all other sequences, the numbering is based on the full-length precursor sequence of the protein. For sequences in which a structure of the ligand-receptor complex has been determined experimentally (e.g. TNFB, TRAIL, BLyS), or is readily modeled (e.g. TNFA), position numbers that lie at a ligand-receptor interface are highlighted in gray. Positions highlighted for RANKL have been experimentally determined to affect receptor binding. Receptor interfaces, highlighted in black, are used to define 7 general receptor contact regions of the TNF superfamily ligands. A generic numbering system, beginning with position number 1, is also included above the MSA for reference.

FIG. 4A is a chart showing that TNF-alpha variants are pre-exchanged with wild-type TNF-alpha to reduce TNF-alpha induced activation of NFkB in 293T cells. FIG. 4B are photographs of the immuno-localization of NFkB in HeLa cells showing that the exchange of wild-type TNFalpha with the A145/Y87H TNFSF variant inhibits TNFSF-induced nuclear translocation of NFkB in HeLa cells. FIG. 4C depicts the TNFSF variant A145R/Y87H reduces wild-typeTNFSF-induced activation of the NFkB-driven luciferase reporter.

FIG. 5 is a chart showing antagonist activity of TNF-alpha variants.

FIGS. 6A-C are dose response curves of caspase activation by various TNF-alpha variants.

FIG. 7A shows a model for the dominant-negative inhibitory RANKL variant strategy. The RANKL variant homotrimers and heterotrimers with wild-type RANKL do not activate the RANK receptor. The RANKL variants act in a dominant-negative manner by rendering the wild-type RANKL protein inactive through the formation of these heterotrimer species. The bumps and sticks on the engineered RANKL protein symbolize the rationally designed mutations in the small and large binding domains of RANKL that interfere with receptor binding. Through this mechanism of inactivating wild-type RANKL, the dominant-negative inhibitory RANKL variants will exert their therapeutic effects.

FIG. 7B shows the list of computationally designed inhibitory RANKL variants. These inhibitory variants can be combined with RANKL solubility variants to produce soluble human RANKL variants.

FIG. 7C shows RANKL solubility variant C22S/1247E mediated osteoclastogenesis is antagonized by OPG and RANK-Fc. The plots show that commercial RANKL from BioSource is antagonized to the same extent as Xencor solubility variant. The BSA and Enbrel controls show that the antagonism is specific to RANKL interacting with OPG and RANK-Fc. The RANK-Fc and OPG controls show that they do not mediate osteoclastogenesis.

FIG. 7D shows aRANKL variant antagonism screen. The ability of RANKL variants to antagonize osteoclastogenesis was evaluated by monitoring TRAP levels when RANKL-C221S/1247E is mixed with RANKL variants, incubated and added to RAW264.7 cells. TRAP is released from RAW264.7 cells as they undergo RANK-mediated osteoclastogenesis. This experiment identified RANKL variants that antagonized this process. FIG. 7E. RANKL variant antagonism screen using a fixed ratio of RANKL-C221S/1247E to ten fold excess variants. The ability of RANKL variants to antagonize osteoclastogenesis was evaluated by monitoring TRAP levels when RANKL-C221S/1247E is mixed with RANKL variants, incubated, diluted over 2 logs, and added to RAW264.7 cells. TRAP is released from RAW264.7 cells as they undergo RANK-mediated osteoclastogenesis. This experiment identified RANKL variants that antagonized this process. FIG. 7F shows a RANKL variant antagonism screen. The ability of RANKL variants to antagonize osteoclastogenesis was evaluated by monitoring TRAP levels when RANKL-C221 S/1247E and RANKL variants are added to RAW264.7 cells without pre-incubation. TRAP is released from RAW264.7 cells as they undergo RANK-mediated osteoclastogenesis. This experiment identified RANKL variants that antagonized this process. FIG. 7G shows a summary of RANKL antagonizing variants. These variants were shown to antagonize RANKL mediated osteoclastogenesis.

### DETAILED DESCRIPTION OF THE INVENTION

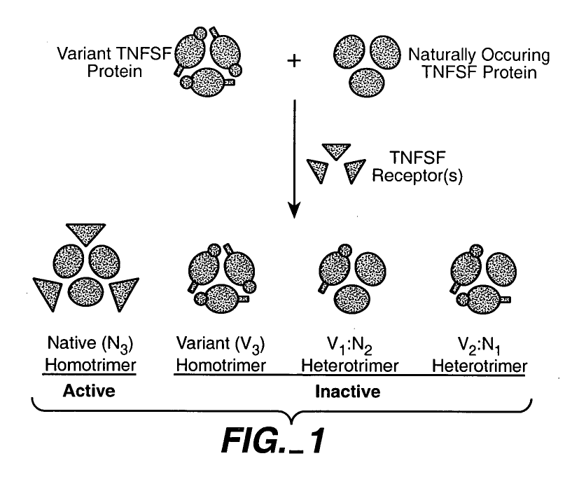

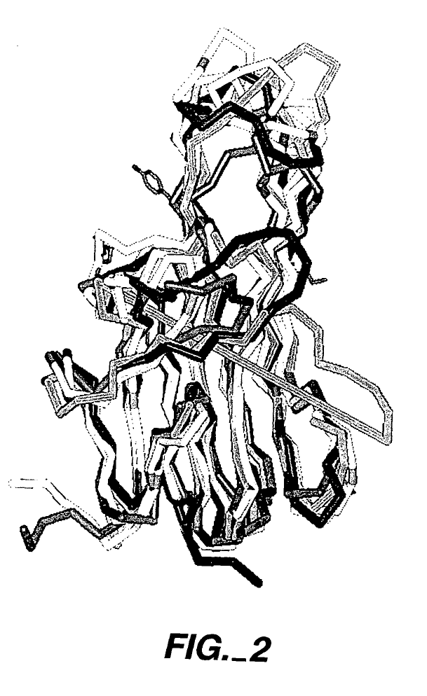

The present invention is directed to novel proteins that exhibit a dominant negative phenotype or mechanism of action. The dominant-negative therapeutic strategy is based on the design of novel TNFSF variants that have reduced receptor binding and/or activation properties as compared to naturally occurring TNFSF proteins, and the ability to oligomerize with naturally occurring TNFSF proteins (FIG. 1). In other words, TNFSF variants that result in reduced activation (preferably do not substantially activate) of TNFSF receptors (as compared to a naturally occurring TNFSF protein) will exchange with at least one naturally occurring TNFSF protein and sequester it into reduced or inactive hetero-oligomers, inhibiting the oligomer's biological activity, e.g. the ability to bind and/or activate the receptor.

The dominant-negative TNFSF variants of the present invention may be designed by modifying TNFSF proteins at key receptor contact points in order to disrupt the ability of the ligand to either bind or activate the receptor. The exchange and physical interaction of these oligomeric TNFSF variants with naturally occurring TNFSF proteins results in reduced activity or deactivation of the naturally occurring TNFSF proteins. To help accomplish this goal more effectively, the TNFSF variants can also be designed to preferentially hetero-oligomerize with naturally occurring TNFSF proteins. Alternatively, the variants may be designed to bind each other and “swamp” out the effect of any naturally occurring TNFSF proteins due to the amount of variant oligomers present; e.g. equilibria favors the binding of the variant mixed oligomers.

Accordingly, the present invention provides methods and compositions utilizing variants of an extracellular domain of a TNFSF protein that antagonize a naturally occurring TNFSF protein.

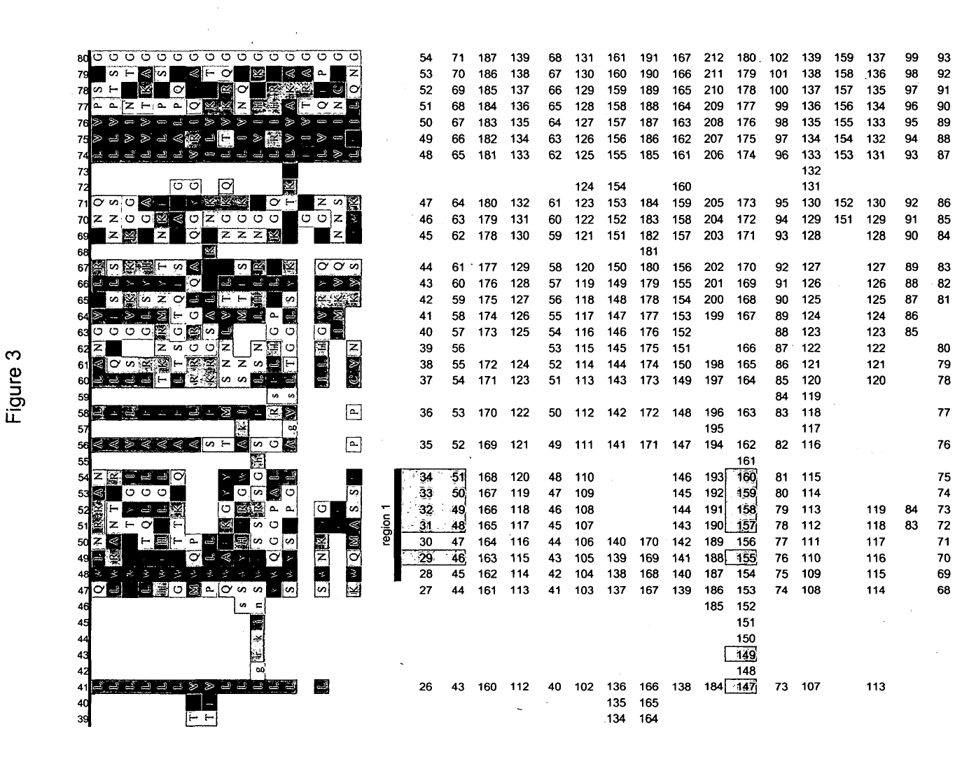

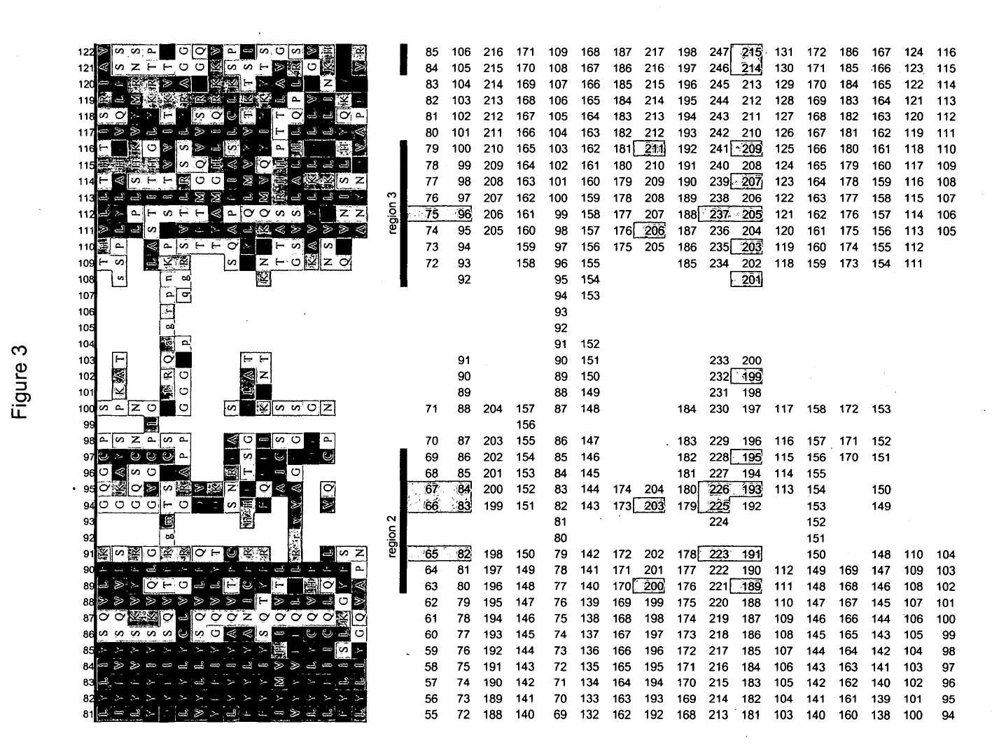

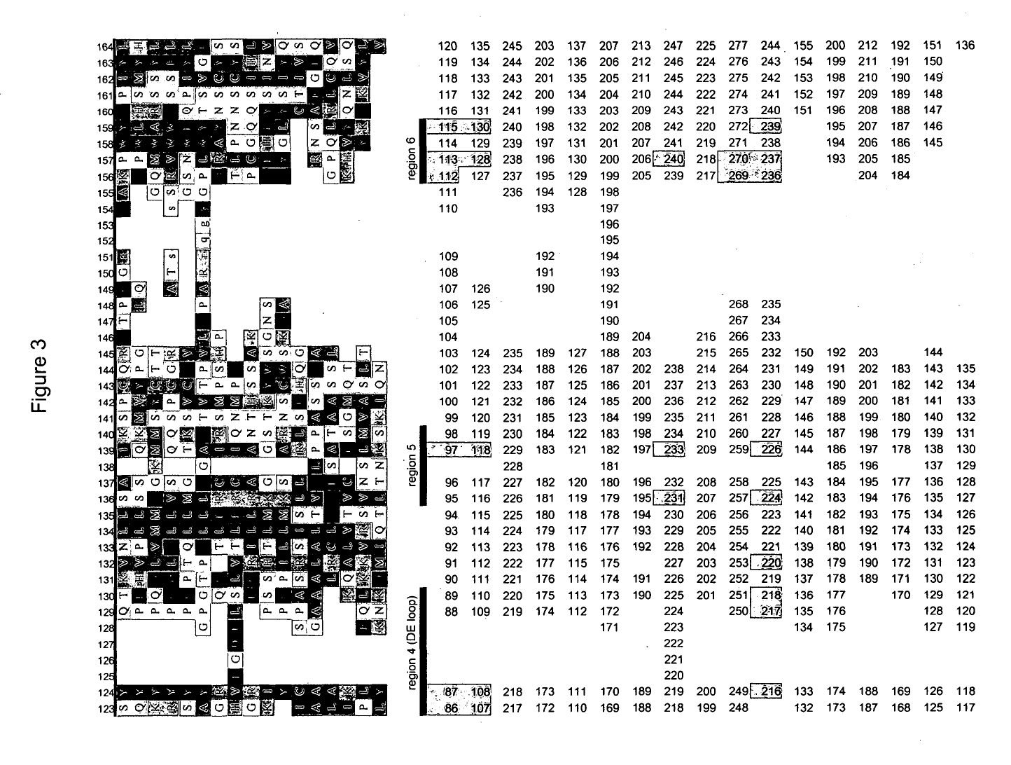

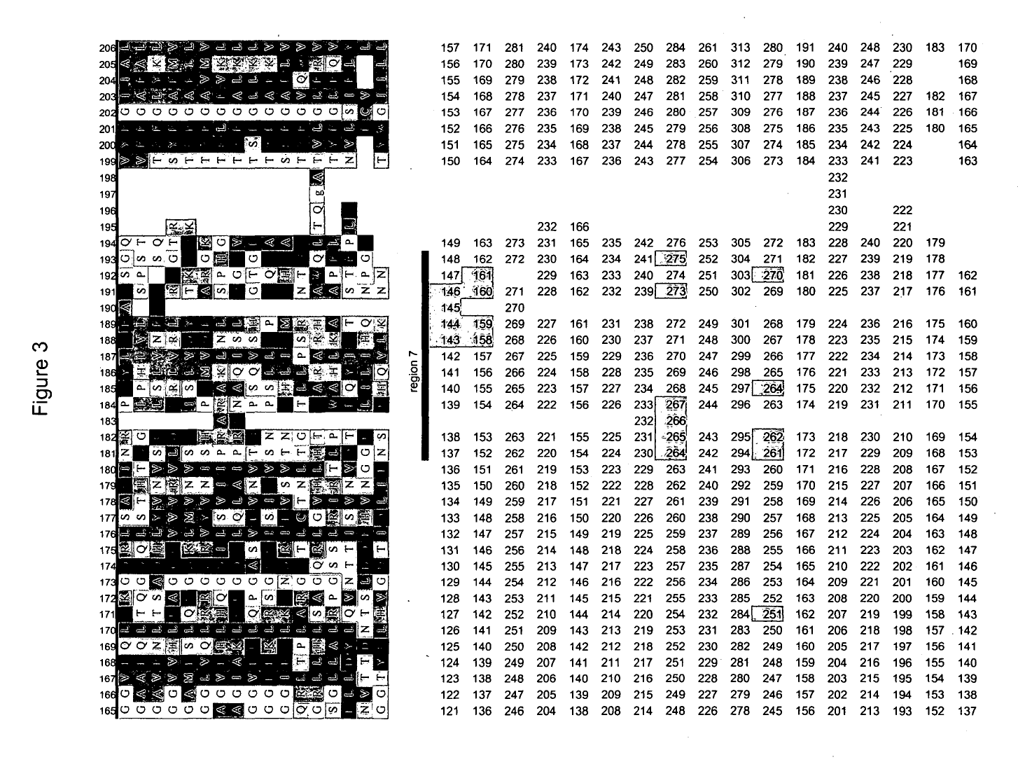

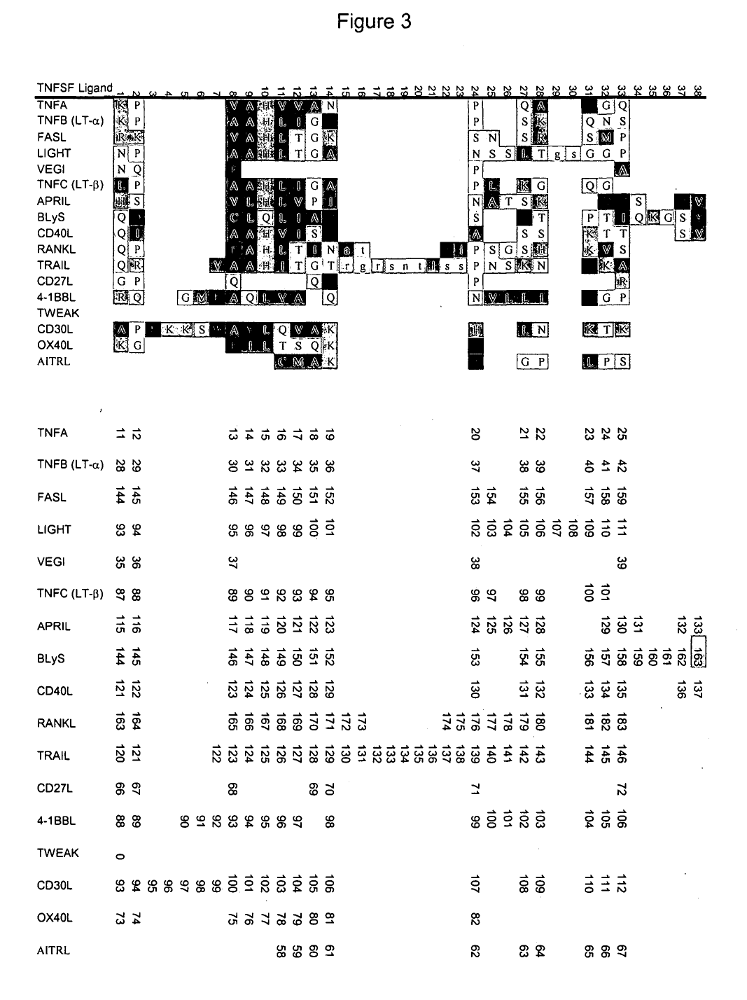

By “extracellular domain” or “ECD” as used herein is meant the segment of protein existing predominantly outside the cell, generally soluble when cleaved or isolated away from the rest of the protein. For transmembrane proteins, this segment can be tethered to the cell through a transmembrane domain or released from the cell through proteolytic digestion. Alternatively, the extracellular domain could comprise the whole protein or amino acid segments thereof when secreted from the cell. In general, TNFSF members are expressed as type 11 transmembrane proteins (extracellular C terminus). The unprocessed protein generally contains an atypical signal anchor/intracellular domain of about 10 to 80 amino acids. The extracellular region may be about 140-215 amino acids in length. Soluble forms of TNFSF proteins may result from proteolytic cleavage of the signal propeptide by matrix metalloproteinases termed TNF-alpha converting enzymes (TACE) or directly by recombinant methods. FIG. 3 depicts a number of extracellular domains from a number of different TNFSF proteins. As will be appreciated by those in the art, these domains may be shorter or longer than those depicted in FIG. 3.

In a preferred embodiment, the extracellular domain can be defined functionally, as a TNFSF protein. or variant protein that is soluble and will form oligomers, preferably with wild-type monomers.

Unless otherwise disclosed, the variant TNFSF proteins of the present invention are composed of the extracellular domain or functional equivalents thereof. That is, the variants of the present invention do not comprise transmembrane domains unless specifically noted. In certain embodiments of the present invention, the variant TNFSF proteins antagonize the membrane bound naturally occurring form of a TNFSF protein and in other embodiments, the variant TNFSF proteins antagonize the soluble form of a naturally occurring TNFSF protein, or both.

The TNFSF proteins of the present invention are variant proteins. The variant TNFSF proteins and nucleic acids of the invention are distinguishable from naturally occurring or wild-type TNFSF. By “naturally occurring”, “wild-type”, “native”, or grammatical equivalents, herein is meant an amino acid sequence or a nucleotide sequence that is found in nature and includes allelic variations; that is, an amino acid sequence or a nucleotide sequence that usually has not been intentionally modified. Accordingly, by “non-naturally occurring” or “synthetic” or “recombinant” or grammatical equivalents thereof, herein is meant an amino acid sequence or a nucleotide sequence that is not found in nature; that is, an amino acid sequence or a nucleotide sequence that usually has been intentionally modified. By the term “recombinant nucleic acid” herein is meant nucleic acid, originally formed in vitro, in general, by the manipulation of nucleic acid by endonucleases, in a form not normally found in nature. Thus an isolated variant TNFSF nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Similarly, a “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild-type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of a variant TNFSF protein from one organism in a different organism or host cell. Alternatively, the protein may be made at a significantly higher concentration than is normally seen, through the use of an inducible promoter or high expression promoter, such that the protein is made at increased concentration levels. Furthermore, all of the variant TNFSF proteins outlined herein are in a form not normally found in nature, as they contain amino acid substitutions, insertions and deletions as compared to the corresponding wild-type (endogeneous) sequence, with substitutions being preferred.

Representative amino acid sequences of naurally occurring human TNFSF are shown in FIG. 3. It should be noted, that unless otherwise stated, all positional numbering of variant TNFSF proteins and variant TNFSF nucleic acids is based on these sequences. That is, as will be appreciated by those in the art, an alignment of TNFSF proteins and variant TNFSF proteins may be done using standard programs, as is outlined below, with the identification of “equivalent” positions between the two proteins. Thus, the variant TNFSF proteins and nucleic acids of the invention are non-naturally occurring; that is, they do not exist in nature.

In a preferred embodiment, the variant TNFSF protein comprises non-conservative modifications (e.g. substitutions). By “nonconservative” modification herein is meant a modification in which the wild type residue and the mutant residue differ significantly in one or more physical properties, including hydrophobicity, charge, size, and shape. For example, modifications from a polar residue to a nonpolar residue or vice-versa, modifications from positively charged residues to negatively charged residues or vice versa, and modifications from large residues to small residues or vice versa are nonconservative modifications. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine. In a preferred embodiment, the variant TNFSF proteins of the present invention have at least one nonconservative modification.

Conservative modifications are generally those shown below, however, as is known in the art, other substitutions may be considered conservative:

| | |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Modifications of the proteins are preferably substitutions and may include those to surface, boundary and core areas of a TNFSF member. See, for example, U.S. Pat. Nos. 6,188,965 and 6,269,312, hereby incorporated by reference. In another preferred embodiment, modifications may be made to surface residues, particularly when alterations to binding properties are desired (either to other monomers or to the receptor).

The variant proteins may be generated, for example, by using a PDA™system previously described in U.S. Pat. Nos. 6,188,965; 6,296,312; 6,403,312; U.S. Ser. Nos. 09/419,351, 09/782,004, 09/927,790, 09/877,695, and 09/877,695; alanine scanning (see U.S. Pat. No. 5,506,107), gene shuffling ((WO 01/25277), site saturation mutagenesis, mean field, sequence homology, or other methods known to those skill in the art that guide the selection of point mutation sites and types.

In a preferred embodiment, sequence and/or structural alignments may be used to generate the variant TNFSF proteins of the invention. As is known in the art, there are a number of sequence-based alignment programs; including for example, Smith-Waterman searches, Needleman-Wunsch, Double Affine Smith-Waterman, frame search, Gribskov/GCG profile search, Gribskov/GCG profile scan, profile frame search, Bucher generalized profiles, Hidden Markov models, Hframe, Double Frame, Blast, Psi-Blast, Clustal, and GeneWise. There are also a wide variety of structural alignment programs known. See for example VAST from the NCBI

(ncbi.nlm.nih.gov:80/Structure/VAST/vast.shtml); SSAP (Orengo and Taylor, Methods Enzymol 266(617-635 (1996)) SARF2 (Alexandrov, Protein Eng 9(9):727-732. (1996)) CE (Shindyalov and Bourne, Protein Eng 11(9):739-747. (1998)); (Orengo et al., Structure 5(8):1093-108 (1997); Dali (Holm et al., Nucleic Acid Res. 26(1):316-9 (1998), all of which are incorporated by reference).

The methods of the present invention can be applied to any recognized member of the TNFSF or related (e.g. the c1q family of proteins) system in which individual domains oligomerize to form an active complex. These domains can be modified in a number of ways to remove or reduce receptor binding and/or activation. In addition, each modified domain can be covalently coupled to at least one additional modified domain to generate dominant negative proteins with enhanced antagonistic activity.

In a preferred embodiment, the proteins belong to the TNFSF (See Oren, D. A., et al., (2002) Nature Structural Biology, 9, 288-292; Bodmer, J-L., et al., (2002) TIBS, 27, 19-26; Locksley, R. M., et al., (2001) Cell, 104, 487-501; WO 01/25277; all of which are expressly incorporated herein by reference). Thus, the definition of “TNFSF” proteins include, but are not limited to, members of the TNFSF of interest include the ligands for TNF; osteoprotegerin (OPG) also known as RANKL (U.S. Pat. No. 5,843,678, incorporated herein by reference), CD40 ligand, BLyS, etc. and others shown in FIG. 3. However, in some embodiments, the TNFSF specifically excludes TNF-α protein.

The compositions and methods of the present invention may also be applied to structural homologues of the TNFSF, including for example, the C1q-related family of proteins, examples of which include the adipocyte complement-related protein of 30 kDa (ACRP30) and its human ortholog APM-1. ACRP30 belongs to a C1 q-related family of proteins comprised of at least 23 members either with or without collagen domains. Those with a collagenous domain include ACRP30, C1 qA, B, and C chains, hibernation-related proteins in chipmunks (HP-20, 25 and 27), CORS26 and Elastin microfibril interface-located protein (EMILIN). Those lacking the collagen domain include multimerin and the precursors of cerebellins 1 and 3. Many of these proteins—which also form trimers or multimers of trimers—have been implicated in development, and immunological and physiological homeostasis.

As outlined herein, there are a variety of mechanisms that may allow the variant TNFSF proteins to serve as antagonists for the wild-type proteins. In preferred embodiments, the variant proteins form hetero-oligomers with endogeneous wild-type TNFSF proteins which are substantially incapable of activating one or more receptors. That is, as illustrated in FIG. 1, a variant TNFSF protein is preferably modified such that interactions with a receptor molecule are disrupted. Preferably, these modifications would not substantially affect the ability of the variant domain to interact with and sequester the naturally occurring TNFSF protein. In a preferred embodiment, these modifications may be combined with additional modifications that enhance the ability of variant TNFSF proteins to hetero-oligomerize with one or more naturally occurring TNFSF proteins. Most preferably, modifications that affect receptor activation and oligomerization are also combined with chemical modifications (e.g., glycosylation, PEGylation, fusions, etc.) that improve pharmacokinetic properties, as further outlined below.

More preferably, the present invention is also directed to novel proteins and nucleic acids possessing TNFSF antagonist activity. Thus, variant proteins possessing “antagonist” activity ultimately result in a lack of receptor activation as further defined below. This may be due to variant monomers interacting with wild-type monomers to “antagonize” the wild-type monomers, e.g. such that wild-type monomers are incapable of binding and/or activating at least one receptor. Generally speaking, the mixed oligomers comprising at least one variant monomer and at least one wild-type (e.g. endogeneous) monomer inhibit receptor activation.

In a preferred embodiment, the variant TNFSF proteins are dominant negative proteins. By “dominant negative” phenotype or “mechanism of action” herein is meant a protein comprising at least one variant TNFSF monomer that has reduced affinity and/or altered signaling for a desired receptor such that the protein retains the ability to oligomerize with other receptor interaction domains but the oligomer cannot substantially interact and/or signal with the desired receptor (see FIG. 1). Depending on the composition of the oligomeric ligand complex, i.e., 2 variants:1 native or 1 variant:2 native for heterotrimers, the degree to which ligand mediated receptor activation is inhibited will vary (see FIG. 1). In other words, receptor activation may be completely inhibited in a complex comprising 2variant:1 native, whereas activation may be reduced in complexes comprising other ratios of variant:native. Alternatively, receptor activation may not be completely inhibited but decreased, again to varying degrees based on the composition of the heterodimer and its corresponding mode of action. See also Menart, V., et al., (2000) Eur J Physiol., 439, R113-R115; U.S. Pat. Pub. Nos. 2002/0039588, 2002/0040132, 2002/0037286, 2002/0037280; all of which are incorporated herein by reference. Monte carlo simulations of heterotrimer assembly, as a function of the relative concentration of variant TNFSF to naturally occurring TNFSF, show that, in general, greater than 99% of the naturally occurring TNFSF monomers will be sequestered when a 10-fold excess of variant TNFSF monomer is added.

In a preferred embodiment, the variant TNFSF proteins of the invention have selectively reduced affinity for one or more receptors while maintaining affinity and/or signaling through alternative receptors. For example, if the desired effect of a dominant-negative variant TNFSF is to reduce activation of a cell type, ideal variants will have reduced signaling through activating receptors but maintained or enhanced signaling through inhibitory receptors.

As will be appreciated by those of skill in the art, two general approaches for creating the dominant negative variants of the present invention include: (1) modifying individual receptor interaction domains to reduce or eliminate receptor binding and/or signaling; and, (2) covalently coupling modified receptor interaction domains to enhance inhibition of receptor activation.

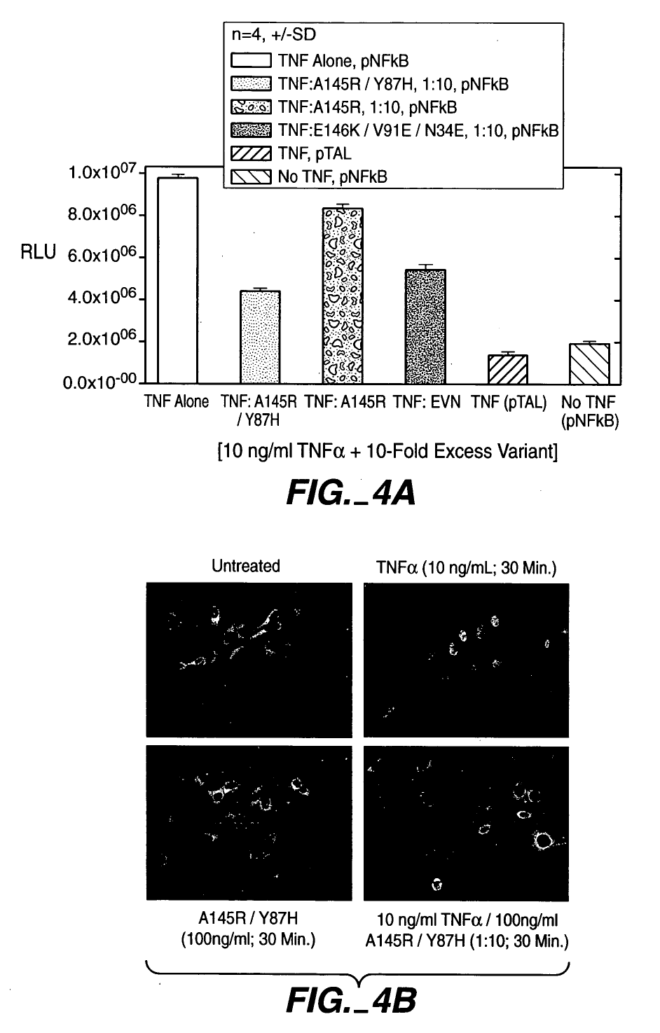

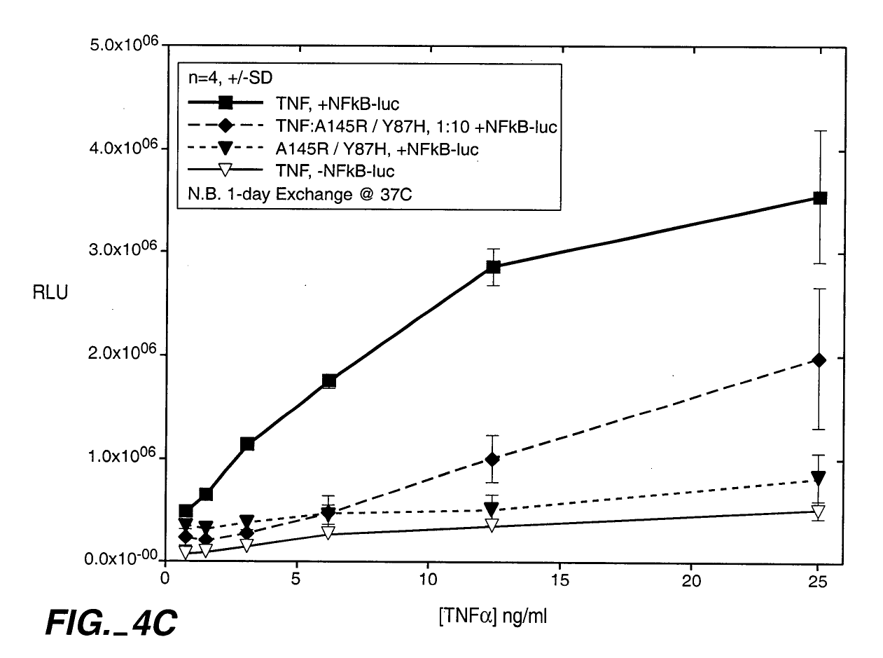

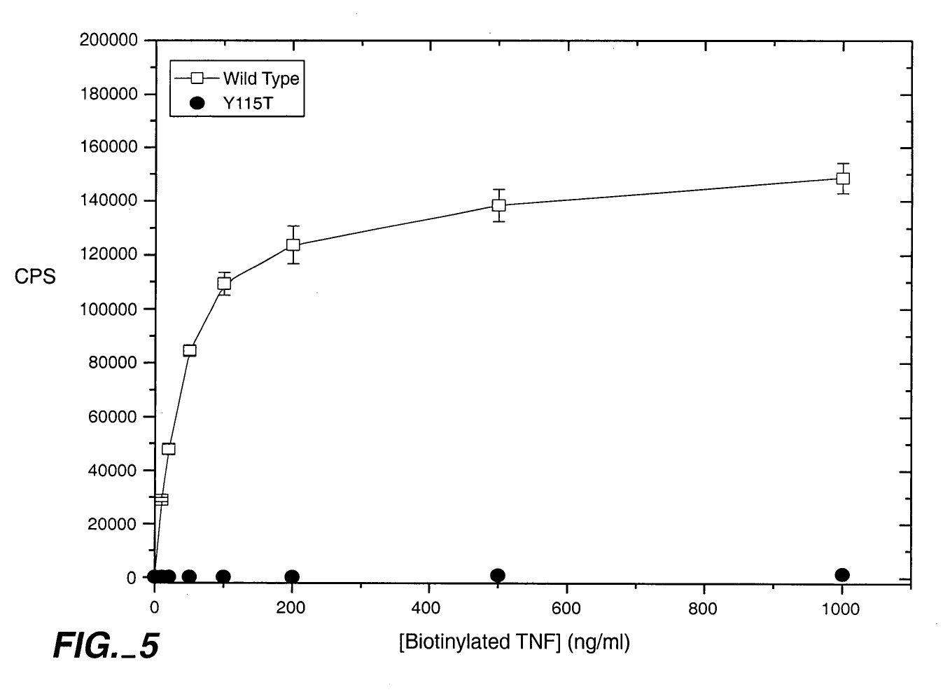

In a preferred embodiment, individual TNFSF proteins are modified within their receptor contact domains to reduce or eliminate receptor binding andior signaling. For example, amino acid substitutions can be generated as modifications in the receptor contact domains that reduce or eliminate receptor binding. In a preferrred embodiment, at least one non-conservative variant in receptor contact domains may be made to disrupt receptor interactions. See FIGS. 4A and 4B, and U.S. Pat. No. 5,506,107; U.S. Ser. Nos. 09/798,789; 09/981,289; 10/262,630; 60/374,035; and 10/338,785; and PCT 03/00393, all of which are hereby incorporated by reference.

Preferable modifications (e.g. substitutions, insertions, deletions, etc.) that affect receptor binding or signaling may be identified using a variety of techniques, including structural alignment methods, sequence alignment methods, etc., as described above. In many cases, the amino acids in the TNFSF ligand that interact with the receptor can be identified directly from a three-dimensional structure of the TNFSF ligand-receptor complex. Alternatively, equivalent information can be derived by analysis of the ligand-receptor complex of a related protein. For example, within the TNFSF, members of the TNF family may be structurally aligned with a TNFSF protein whose structure has been determined experimentally (see for example, Oren, D. A., et al., (2002) Nature Structural Biology, 9, 288-292). Alternatively, if it is not possible to structurally align residues, sequence alignment may be used.

As is known in the art, there are a number of sequence alignment methodologies that may be used. For example, sequence homology based alignment methods may be used to create sequence alignments of TNFSF members (Altschul et al., J. Mol. Biol. 215(3): 403-410 (1990); Altschul et al., Nucleic Acids Res. 25:3389-3402 (1997), both incorporated by reference).

In a preferred embodiment, as highlighted in FIG. 3, the amino acid sequences of members of the TNF superfamily may be aligned into a multiple sequence alignment (MSA). The alignment shown in FIG. 3 was derived originally from the Pfam database, and then further manipulated according to structural alignment (using CE) of the crystal structures of TNFA, TNFB, CD40L, TRAIL, and BLyS. The MSA may also be used to extend the known structural information for additional recognized TNFSFmembers and other structural homologues and families. Due to the high extent of structural homology between different TNFSF members, the MSA may be used as a reliable predictor of the effects of modifications at various positions within the alignment. For this, the TNFA sequence and numbering shown in FIG. 3 can be used as an MSA reference point for any other TNFSF protein sequence. As used herein, referral to “TNFSF protein positions corresponding to TNFA amino acid X”, represents referral to a collection of equivalent positions in other recognized TNFSF members and structural homologues and families. For example, TNFSF protein positions corresponding to TNFA amino acid L75 corresponds to the following amino acid positions in the following TNFSF proteins: TNFA:L75, TNFB:Y96, FASL:P206, LIGHT:T161, VEGI:S99, Lymphotoxin beta:T158, APRIL:T177, BLyS:A207, CD40L:P188, RANKL:Q237, TRAIL:Q205, CD27L:S121, 4-1BBL:S162, TWEAK:Y176, CD30L:D157, OX40L:N114, AITRL:N106, and equivalent positions in the other proteins recognized as TNFSF members.

For example, analysis of a structure of the complex of TNFB with the p55 (R1) receptor indicates that the amino acid Y108 in TNFB directly contacts the receptor. The analogous residue Y216 from TRAIL also directly contacts the DR5 receptor. The MSA thus predicts that the analogous residue 197 from TNFA also contacts a receptor. Consistent with this prediction, mutation of TNFA-197 to R or T results in a significant loss of receptor-binding affinity and biological signaling activity. The analysis for this contact position can be extended to all members of the family, predicting that the following positions are important for receptor interactions: FASL:Y218, LIGHT:Y173, VEGI:Y111, TNFC:Y170, APRIL:R189, BlyS:V219, CD40L:R200, RANKL:1249, CD27L:C133, 4-1BBL:A174, TWEAK:A188, CD30L:K169, OX40L:L126, and AITRL:Y118. This kind of analysis can be performed for all receptor contact regions of the ligands.

FIG. 3 highlights 7 canonical receptor contact regions based on analysis of known structures and mutational data. In preferred embodiments of the invention, each of the 7 regions highlighted in FIG. 3 as a receptor-contact region is used to define modification sites for the creation of variants of each TNFSF member. In additional preferred embodiments, such modifications reduce receptor affinity and/or signaling capacity. In additional preferred embodiments, these modifications also preserve the ability of each protein to oligomerize with naturally occurring TNFSF proteins, including, but not necessarily limited to, the corresponding wild-type sequence of each family member.

Using the alignment system depicted in FIG. 3 or other alignment programs discussed above, one can use as a reference point, the numbering system of any alignment program and may correlate the relevant positions of the TNFA protein with equivalent positions in other recognized members of the TNFSF or structural homologues and families.

For purposes of the present invention, the areas of TNFSF proteins to be modified are preferably but not required to be selected from the group consisting of the Large Domain, Small Domain, the DE loop, and the trimer interface. The Large Domain, the Small Domain and the DE loop are three separate receptor contact domains, each made up of several non-contiguous linear segments of the protein (i.e. the 7 canonical receptor contact regions described above). These domains are identified in the TNFSF proteins—and the MSA—by comparison to the receptor interaction domains of Lymphotoxin-alpha and TRAIL, two TNFSF proteins whose structures (PDB entries 1TNR and 1D0G, respectively) have been defined in complex with their cognate receptors using crystallographic methods. The trimer interface mediates interactions between individual TNFSF protein monomers. Trimerization positions can be identified either directly from the crystal structure of the appropriate TNFSF protein (e.g. for TNFA, TNFB, BLyS, TRAIL, CD40L, or RANKL), or by analogy to another TNFSF protein. In a preferred embodiment, positions from one TNFSF protein monomer containing atoms that are within 5 angstroms distance from a neighboring monomer are designated as trimer interface positions. Modifications may be made solely in one of these areas or in any combination of these and other areas.

The Large Domain preferred positions to be modified in TNFSF proteins include but are not limited to TNFA corresponding positions 28-34, 63-69, 112-115, and 137-147. For the Small Domain, the preferred positions to be modified include but are not limited to TNFA corresponding positions 72-79 and 95-98. For the DE Loop, the preferred positions to be modified include but are not limited to TNFA corresponding positions 84-89. The Trimer Interface positions to be modified include but are not limited toTNFA corresponding positions 11, 13, 15, 34, 36, 53-55, 57, 59, 61, 63, 72, 73, 75, 77, 119, 87, 91-99, 102-104, 109, 112-125, 147-149, 151, and 155-157. Especially preferred trimer interface positions to be modified are TNFA corresponding positions 57, 34, and 91. For example, amino acids X and Y at TNFA corresponding positions 34 and 91 can be replaced simultaneously by similarly charged amino acids (e.g. X34E+Y91E, X34K+Y91R, etc.) to generate electrostatic repulsion at the variant monomer-monomer interfaces while not perturbing the stability of variant-native interfaces.

In a preferred embodiment, the choice of modification site and type is made by referring to other sequences in the alignment. Thus, in a preferred embodiment, the original amino acid X from sequence A is mutated to amino acid Y from sequence B, such that Y is a nonconservative substitution relative to amino acid X. For example, the amino acid Y87 from TNFA aligns with the non-conservative R189 from APRIL. Indeed, as previous studies have shown, the Y87R substitution in TNFA leads to a significant decrease in receptor binding and signaling by TNFA. In additional embodiments, more conservative mutations can also be utilized. In additional embodiments, the wild-type residue is mutated to alanine.

In a preferred embodiment, useful modifications at receptor contact and/or trimerization interfaces are selected using protein design or modeling algorithms such as PDA™ technology (see, U.S. Pat. No. 6,188,965; 6,269,312; 6,403,312; U.S. Ser. No. 09/714,357; 09/812,034; 09/827,960; 09/837,886; 09/782,004 and 10/218,102, all hereby incorporated by reference). As is known in the art, algorithms in this class generally use atomic-level or amino acid level scoring functions to evaluate the compatibility of amino acid sequences with the overall tertiary and quaternary structure of a protein. Thus, algorithms of this class can be used to select receptor-binding disruptions that do not substantially perturb the ability of variant TNFSF proteins to properly fold and oligomerize with themselves or their naturally occurring targets. These technologies typically use high-resolution structural information of the target protein as input. In a preferred embodiment, an experimentally determined strucure of the appropriate TNFSF protein is used as input. In alternative embodiments, the MSA can be used to guide the construction of atomic-level homology models for TNFSF members based on the subset of the family whose three-dimensional structures have been determined using crystallographic or related methods. For the TNFSF, high-resolution structures have been determined for TNFA, TNFB (LT-alpha), TRAIL, CD40L, and BLyS. The homology models can in turn be used as structural scaffolds to guide the design of variant TNF superfamily ligands that possess reduced receptor binding and/or signaling and/or dominant-negative activity.

In alternative embodiments, protein design algorithms may be used to generate mutations in individual receptor interaction domains that create steric repulsion between the receptor interaction domain and the receptor. Other mutations that may be generated include, but are not limited to, mutations that create electrostatic repulsion, and mutations that create unfavorable desolvation of amino acids.

In a preferred embodiment, substitutions, insertions, deletions or other modifications at multiple receptor interaction and/or trimerization domains may be combined. Such combinations are frequently advantageous in that they have additive or synergistic effects on dominant-negative activity. Examples include, but are not limited to, simultaneous substitution of amino acids at the large and small domains (e.g. MSA positions 94 and 112), large domain and DE loop (e.g. MSA positions 95 and 124), large domain and trimerization domain (e.g. MSA positions 94 and 83), or multiple substitutions within a single domain. Additional examples include any and all combinations of substitutions.

In preferred embodiments, the defined receptor contact regions constitute sites for insertion, deletion, or substitution of amino acid residues, or sites for the introduction of chemical modification sites. In a preferred embodiment, deletions or insertions are made in accordance with the MSA. For example, inspection of the MSA reveals that BLyS amino acids 220-223 constitute a 4-residue insertion relative to many of the additional family members. This region lies in the DE-loop region of the protein, and is known to contribute to the formation of higher-order oligomers of BLySThus, deletion of these four residues, while predicted to maintain the structural integrity of the BLyS protein, is expected to reduce the propensity of BLyS to form higher-order (i.e. higher than trimer) oligomers.

In additional embodiments, the variants described above can be combined with other modifications to the TNF superfamily ligand. These include, but are not limited to, additional amino acid substitutions, insertions, or deletions, and/or chemical (e.g. PEGylation) or posttranslational modifications such as glycosylation (see WO 99/45026; WO 01/49830; WO 01/49830; WO 02/02597; WO 01/58493; WO 01/51510, U.S. Pat. Nos. 4,002,531; 5,183,550; 5,089,261; 6,153,265; 5,264,209; 5,383,657; 5,766,897; 5,986,068; 4,280,953; 5,089,261; 5,990,237; 6,461,802; 6,495,659; 6,448,369; 6,437,025; 5,900,461; 6,413,507; 5,446,090; 5,672,662; 5,919,455; 6,113,906; 5,985,236; 6,214,966; 6,258,351; 5,932,462; EP 0786 257; EP 0 902 085; EP 1 064 951; EP 0 544 826; EP 0 424 405; EP 0 400 472; EP 0 311 589;; Veronese, F. M. (2001) Biomaterials, 22: 405-471; all of which are incorporated herein by reference). In some embodiments, for example in the creation of animal models of disease, fusion proteins comprising the variant TNFSF proteins with other sequences may be done, for example using fusion partners comprising labels (e.g. autofluorescent proteins, survival and/or selection proteins), stability and/or purification sequences, toxins, variant proteins from other members of the superfamily (e.g. analogous to the creation of “bi-specific antibodies”) or any other protein sequences of use. Additional fusion partners are described below. In some instances, the fusion partner is not a protein.

In a preferred embodiment, additional amino acid substitutions are made to optimize hetero-oligomer interactions between variant ligand and its endogenous counterpart and/or to destabilize the oligomeric state of the variant alone. For example, an L57F mutation in TNFA has been designed in order to promote the formation of variant:native heterotrimers while disfavoring the formation of variant homotrimers. Such modifications are useful for promoting the exchange of variant monomers with native monomers in order to promote a dominant-negative mechanism of action.

As will be understood by those in the art, variant TNF superfamily ligands which have reduced signaling capacity can be discovered by a large variety of methods, including, but not limited to, directed evolution (e.g. error prone PCR, DNA shuffling, etc.), single-site saturation mutagenesis, and alanine-scanning mutagenesis. Furthermore, it is possible that use of these or other methods will allow the discovery of substitutions, insertions, or deletions—which reduce receptor binding and/or signaling activity—that lie outside of the 7 canonical contact regions described herein.

In another embodiment, coiled-coil motifs are used to assist dimer assembly (see Dahiyat et al., Protein Science 6:1333-7 (1997) and U.S. Ser. No. 09/502,984; both of which are incorporated herein by reference in their entirety). Coiled coil motifs comprise, but is not limited to one of the following sequences: RMEKLEQKVKELLRKNERLEEEVERLKQLVGER, based on the structure of GCN4; AALESEVSALESEVASLESEVAAL, and LAAVKSKLSAVKSKLASVKSKLAA, coiled-coil leucine zipper regions defined previously (see Martin et al., EMBO J. 13(22): 5303-5309 (1994), incorporated by reference). Other coiled coil sequences from e.g. leucine zipper containing proteins are known in the art and are used in this invention. See, for example, Myszka et al., Biochem. 33:2362-2373 (1994), hereby incorporated by reference).

Similarly, molecular dynamics calculations can be used to computationally screen sequences by individually calculating mutant sequence scores and compiling a list.

In a preferred embodiment, residue pair potentials can be used to score sequences (Miyazawa et al., Macromolecules 18(3): 534-552 (1985), expressly incorporated by reference) during computational screening.

As will be appreciated by those in the art, additional TNFSF proteins may be identified and added to the MSA highlighted in Fig. X. The source of the sequences may vary widely, and include taking sequences from one or more of the known databases, including, but not limited to, GenBank (.ncbi.nlm.nih.gov/).

In addition, sequences from these databases may be subjected to contiguous analysis or gene prediction; see Wheeler, et al., Nucleic Acids Res 28(1):10-14. (2000) and Burge and Karlin, J Mol Biol 268(1):78-94. (1997).

As is known in the art, there are a number of sequence alignment methodologies that may be used. For example, sequence homology based alignment methods (Altschul et al., J. Mol. Biol. 215(3): 403-410 (1990); Altschul et al., Nucleic Acids Res. 25:3389-3402 (1997), both incorporated by reference) may be used to create sequence alignments of TNFSF members. These sequence alignments are then examined to determine the observed sequence variations. These sequence variations are tabulated to define a set of variant TNFSF proteins.

Sequence based alignments may be used in a variety of ways. For example, a number of related proteins may be aligned, as is known in the art, and the “variable” and “conserved” residues defined; that is, the residues that vary or remain identical between the family members can be defined. These results may be used to guide the design of variant protein libraries whose properties can be probed experimentally. For example, the positions of high variability between family members (i.e. low conservation) may be randomized, either using all or a subset of amino acids. Alternatively, the sequence variations may be tabulated and appropriate substitutions defined from them. Alternatively, the allowed sequence variations may be used to define the amino acids considered at each position during a computational modeling and/or screening process. Another variation is to bias the score for amino acids that occur in the sequence alignment, thereby increasing the likelihood that they are found during computational screening but still allowing consideration of other amino acids. This bias would result in a focused library of variant TNFSF proteins but would not eliminate from consideration amino acids not found in the alignment.

As used herein variant TNFSF or TNFSF proteins include TNFSF monomers, dimers or trimers, with the former two preferred when combined into trimers comprising wild-type protein.

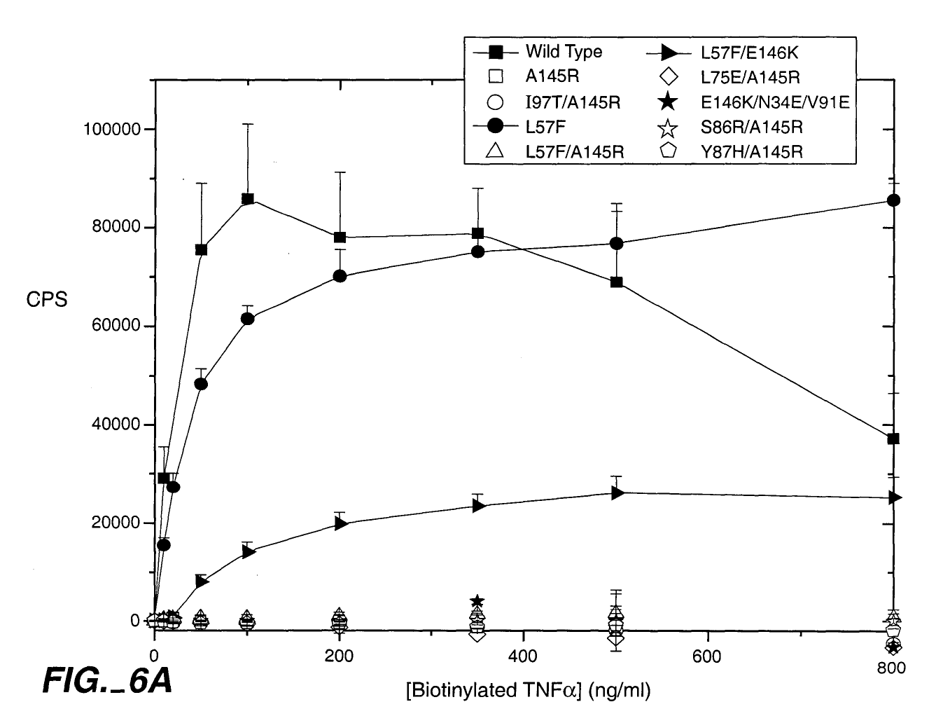

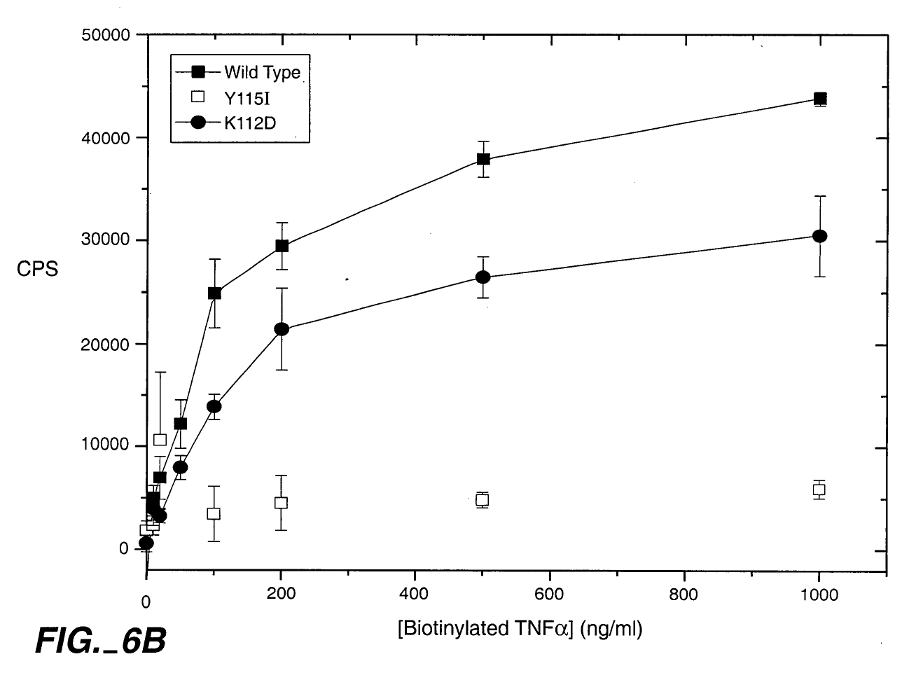

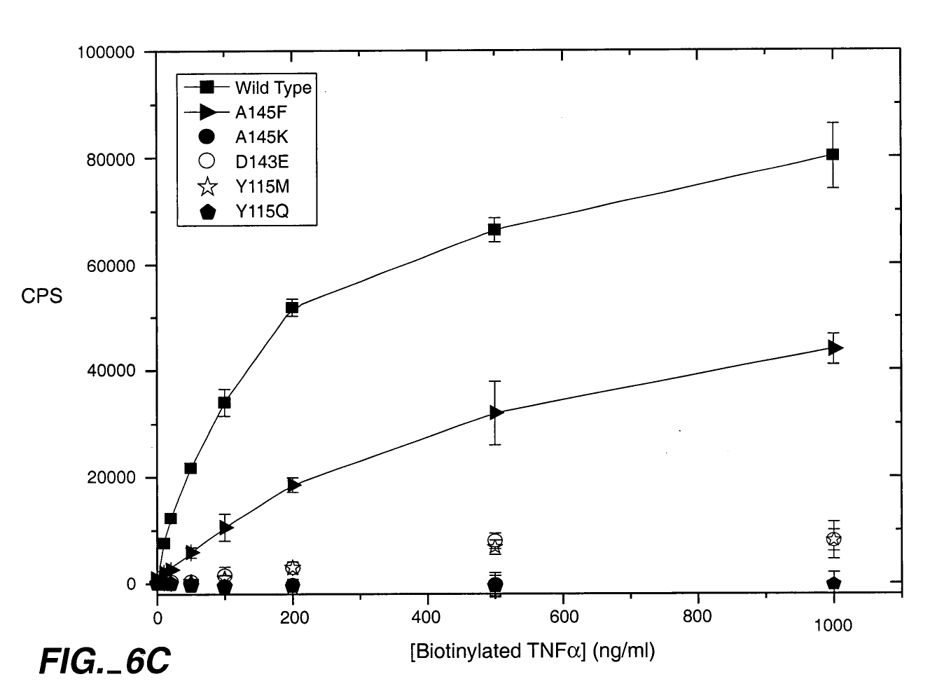

The TNFSF proteins may be from any number of organisms, with TNFSF proteins from mammals being particularly preferred. Suitable mammals include, but are not limited to, rodents (rats, mice, hamsters, guinea pigs, etc.), primates, farm animals (including sheep, goats, pigs, cows, horses, etc); and in the most preferred embodiment, from humans (the sequence of which is depicted in FIG. 6B). As will be appreciated by those in the art, TNFSF proteins based on TNFSF proteins from mammals other than humans may find use in animal models of human disease.

The variant TNFSF proteins of the invention are antagonists of naturally occurring TNFSF proteins. By “antagonists of naturally occurring TNFSF” herein is meant that the variant TNFSF protein inhibits or significantly decreases the activation of receptor signaling as compared to activation by a naturally occurring member of the TNFSF in the absence of the antagonist.

In a preferred embodiment, the variant TNFSF protein physically interacts with its corresponding wild-type TNFSF protein (i.e. the endogeneous naturally occurring protein from which the variant was derived) such that the complex comprising the variant TNFSF and wild-type TNFSF is substantially incapable of activating TNFSF receptors. Preferably, the variant TNFSF protein preferentially interacts with a wild-type TNFSF to form mixed trimers with the wild-type protein such that receptor binding does not occur and/or TNFSF signaling is not initiated (FIG. 1).

In an alternative embodiment, the variant TNFSF protein physically interacts with a non-corresponding wild-type protein (i.e. a naturally occurring protein different from the protein from which the variant was derived). For example, because APRIL is known to form homotrimers with itself and heterotrimers with BLyS, dominant-negative variant APRIL protein can be used to inhibit naturally occurring APRIL or BLyS proteins. Similarly, since lymphotoxin α and ,β can form active heterotrimers with each other, dominant-negative variant LT-α proteins of the invention can be used to inactivate LT-β.

By mixed trimers herein is meant that monomers of native and variant TNFSF proteins interact to form trimeric TNFSF (FIG. 1). Mixed trimers may comprise 1 variant TNFSF protein:2 native TNFSF proteins, 2 variant TNFSF proteins:1 native TNFSF protein. In some embodiments, trimers may be formed comprising only variant TNFSF proteins (FIG. 1B).

In a preferred embodiment, the variant TNFSF antagonist proteins of the invention are highly specific antagonists for the corresponding wild-type TNFSF protein. However, in alternative embodiments, the variant TNFSF antagonistic proteins of the invention are specific for more than one wild-type TNFSF protein. For example, variant APRIL proteins may be specific antagonist of wild-type APRIL only, wild-type APRIL and BLyS, or wild-type BLyS only. Additional characteristics of the variant TNFSF antagonist proteins include improved stability, pharmacokinetics, and high affinity for native TNFSF. Variants with higher affinity toward native TNFSF may be generated from variants exhibiting TNFSF antagonism as outlined above.

In a preferred embodiment, variant TNFSF proteins exhibit decreased biological activity as compared to native TNFSF, including but not limited to, decreased binding to the receptor, decreased activation and/or ultimately a loss of cytotoxic activity or otherwise undesired activities that can lead to deleterious side effects. By “cytotoxic activity” herein refers to the ability of a TNFSF variant to selectively kill or inhibit cells. Variant TNFSF proteins that exhibit less than 75-50% biological activity as compared to native are preferred. More preferred are variant TNFSF proteins that exhibit less than 25%, even more preferred are variant proteins that exhibit less than 15%, and most preferred are variant TNFSF proteins that exhibit less than 10% of a biological activity of a naturally occurring TNFSF. Suitable assays include, but are not limited to, TNFSF cytotoxicity assays, DNA binding assays; transcription assays (using reporter constructs; see Stavridi, supra); size exclusion chromatography assays and radiolabeling/immuno-precipitation; see Corcoran et al., supra); and stability assays (including the use of circular dichroism (CD) assays and equilibrium studies; see Mateu, supra); all of which are expressly incorporated by reference. These assays may utilized labeled variant proteins, with suitable labels including radioisotopes, chromophores (particularly fluorophores), enzymes, particles e.g. magnetic particles, etc.

In one embodiment, at least one property critical for binding affinity of the variant TNFSF proteins is altered when compared to the same property of native TNFSF and in particular, variant TNFSF proteins with altered receptor affinity are preferred. Also preferred are variant TNFSF with altered affinity toward oligomerization to native TNFSF.

Thus, the invention provides variant TNFSF proteins (e.g. mixed oligomers containing variant monomers) with altered binding affinities such that the variant TNFSF monomer proteins will preferentially oligomerize with wild-type (e.g. endogeneous) TNFSF monomers, but do not substantially agonize TNFSF receptors. “Preferentially” in this case means that given equal amounts of variant TNFSF monomers and wild-type TNFSF monomers, at least 10% and more preferably at least 25% of the resulting trimers are mixed trimers of variant and wild-type TNFSF, with at least about 50% being preferred, and at least about 80-90% being particularly preferred. In other words, it is preferable that the variant TNFSF proteins of the invention have greater affinity for wild-type TNFSF protein as compared to wild-type TNFSF proteins. By “do not substantially interact with TNF receptors” herein is meant that the variant TNFSF proteins will not be able to associate with TNFSF receptors to substantially activate the receptors and/or initiate the TNFSF signaling pathway(s). In a preferred embodiment, at least a 10% decrease in receptor activation is seen, with greater than 20%, 50%, 76%, 80-90% being preferred. By “agonize TNFSF receptors” herein is meant that the variant TNFSF protein enhances the activation of receptor signaling. Generally, variant TNFSF proteins that function as agonists of wild-type TNFSF are not preferred.

Variant TNFSF proteins may be experimentally tested and validated using in vivo and in vitro assays. Suitable assays include, but are not limited to, activity assays and binding assays. Screens that may be utilized in identifying TNFSF variants that are antagonists of TNFSF proteins include, but are not limited to, caspase activation, NF-kB nuclear translocation (Wei et al., Endocrinology 142, 1290-1295, (2001)) or c-Jun (Srivastava et al., JBC 276, 8836-8840 (2001)) transcription factor activation assays, B-cell proliferation assays and IgE secretion assays.

In a preferred embodiment, binding affinities for the following interactions are determined and compared: 1) variant TNFSF oligomer formation, 2) wild-type TNFSF oligomer formation, 3) variant TNFSF binding to a cognate receptor, 4) wild-type TNFSF binding to cognate receptor, 5) variant TNFSF binding to decoy receptor, and 6) wild-type TNFSF binding to decoy receptor. Suitable assays include, but are not limited to, quantitative comparisons comparing kinetic and equilibrium binding constants. The kinetic association rate (Kon) and dissociation rate (Koff), and the equilibrium binding constants (Kd) may be determined using surface plasmon resonance on a BlAcore instrument following the standard procedure in the literature [Pearce et al., Biochemistry 38:81-89 (1999)]. Several alternative methods can also be used to determine binding affinity and kinetics, including but not limited to proximity assays such as AlphaScreen™ (Packard BioScience®).

TNFSF variants can also be tested to determine whether they are capable of forming mixed oligomers including but not limited to mixed trimers. In a preferred embodiment, this is accomplished by labeling native TNFSF and variant TNFSF with distinguishable tags, combining native and variant TNFSF, and screening for oligomers that contain both tag types. For example, FLAG-tagged native TNFSF and His-tagged variant TNFSF can be combined, and sandwich ELISAs can be performed to identify trimers that contain both FLAG and His tag. Another alternative is to run native gels to separate the mixture into separate species and detect using coomassie staining or Western blots using both anti-FLAG and anti-His tag antibodies. This method relies on the fact that FLAG and His tags significantly perturb protein migration in native gels. As will be appreciated by those in the art, many alternate protocols could also be used to measure the formation of mixed trimers.

As outlined above, the invention provides variant TNFSF nucleic acids encoding variant TNFSF polypeptides. The variant TNFSF polypeptide preferably has at least one altered property as compared to the same property of the corresponding naturally occurring TNFSF polypeptide. The property of the variant TNFSF polypeptide is the result of the present invention.

The term “altered property” or grammatical equivalents thereof in the context of a polypeptide, as used herein, further refers to any characteristic or attribute of a polypeptide that can be selected or detected and compared to the corresponding property of a naturally occurring protein. These properties include, but are not limited to cytotoxic activity; oxidative stability, substrate specificity, substrate binding or catalytic activity, thermal stability, alkaline stability, pH activity profile, resistance to proteolytic degradation, kinetic association (Kon) and dissociation (Koff) rate, protein folding, inducing an immune response, ability to bind to a ligand, ability to bind to a receptor, ability to be secreted, ability to be displayed on the surface of a cell, ability to oligomerize, ability to signal, ability to stimulate cell proliferation, ability to inhibit cell proliferation, ability to induce apoptosis, ability to be modified by phosphorylation or glycosylation, and the ability to treat disease.

Unless otherwise specified, a substantial change in any of the above-listed properties, when comparing the property of a variant TNFSF polypeptide to the property of a naturally occurring TNFSF protein is preferably at least a 20%, more preferably, 50%, more preferably at least a 2-fold increase or decrease.

A change in cytotoxic activity is evidenced by at least a 75% or greater decrease in cell death initiated by a variant TNFSF protein as compared to wild-type protein.

A change in binding affinity is evidenced by at least a 5% or greater increase or decrease in binding affinity to wild-type TNF receptor proteins or to wild-type TNFSF.

In a preferred embodiment, the antigenic profile in the host animal of the variant TNFSF protein is similar, and preferably identical, to the antigenic profile of the host TNFSF; that is, the variant TNFSF protein does not significantly stimulate the host organism (e.g. the patient) to an immune response; that is, any immune response is not clinically relevant and there is no allergic response or neutralization of the protein by an antibody. That is, in a preferred embodiment, the variant TNFSF protein does not contain additional or different epitopes from the wild-type or naturally occurring TNFSF. By “epitope” or “determinant” herein is meant a portion of a protein which will generate and/or bind an antibody. Thus, in most instances, no significant amounts of antibodies are generated to a variant TNFSF protein in its native host. In general, this is accomplished by not significantly altering surface residues, as outlined below nor by adding any amino acid residues on the surface which can become glycosylated, as novel glycosylation can result in an immune response, nor by the introduction of new MHC binding epitopes.

The variant TNFSF proteins of the present invention may be shorter or longer than the amino acid sequences shown in FIG. 3. As used in this invention, “wild-type TNFSF” is a native mammalian protein (preferably human). TNFSF may be polymorphic. Thus, in a preferred embodiment, included within the definition of variant TNFSF proteins are portions or fragments of the sequences depicted herein. Fragments of variant TNFSF proteins are considered variant TNFSF proteins if a) they share at least one antigenic epitope; b) have at least the indicated homology; c) and preferably have variant TNFSF biological activity as defined herein.

In a preferred embodiment, as is more fully outlined below, the variant TNFSF proteins include further amino acid variations, as compared to a wild-type TNFSF, than those outlined herein. Examples include, but are not limited to, amino acid substitutions introduced to enable soluble expression in E. coli, amino acid substitutions introduced to optimize solution behavior, and amino acid substitutions introduced to modulate immunogenicity. In addition, as outlined herein, any of the variations depicted herein may be combined in any way to form additional novel variant TNFSF proteins.

In addition, variant TNFSF proteins may be made that are longer than those depicted in the figures, for example, by the addition of epitope or purification tags, as outlined herein, the addition of other fusion sequences, etc. For example, the variant TNFSF proteins of the invention may be fused to other therapeutic proteins or to other proteins such as Fc or serum albumin for pharmacokinetic purposes. See for example U.S. Pat. No. 5,766,883 and 5,876,969, both of which are expressly incorporated by reference.

Variant TNFSF proteins may also be identified as being encoded by variant TNFSF nucleic acids. In the case of the nucleic acid, the overall homology of the nucleic acid sequence is commensurate with amino acid homology but takes into account the degeneracy in the genetic code and codon bias of different organisms. Accordingly, the nucleic acid sequence homology may be either lower or higher than that of the protein sequence, with lower homology being preferred.

In a preferred embodiment, a variant TNFSF nucleic acid encodes a variant TNFSF protein. As will be appreciated by those in the art, due to the degeneracy of the genetic code, an extremely large number of nucleic acids may be made, all of which encode the variant TNFSF proteins of the present invention. Thus, having identified a particular amino acid sequence, those skilled in the art could make any number of different nucleic acids, by simply modifying the sequence of one or more codons in a way which does not change the amino acid sequence of the variant TNFSF.

The variant TNFSF proteins and nucleic acids of the present invention are preferably recombinant (unless made synthetically). As used herein, “nucleic acid” may refer to either DNA or RNA, or molecules which contain both deoxy- and ribonucleotides. The nucleic acids include genomic DNA, cDNA, mRNA and oligonucleotides including sense and anti-sense nucleic acids. Such nucleic acids may also contain modifications in the ribose-phosphate backbone to increase stability and half-life of such molecules in physiological environments.

The nucleic acid may be double stranded, single stranded, or contain portions of both double stranded or single stranded sequence. As will be appreciated by those in the art, the depiction of a single strand (“Watson”) also defines the sequence of the other strand (“Crick”); thus the sequence depicted in FIG. 6 also includes the complement of the sequence.

Also included within the definition of variant TNFSF proteins of the present invention are amino acid sequence variants of the variant TNFSF sequences outlined herein and shown in the Figures. That is, the variant TNFSF proteins may contain additional variable positions as compared to human TNFSF other than those used to generate dominant negative proteins. As for “dominant negative variable positions”, these variants fall into one or more of three classes: substitutional, insertional or deletional variants. All variants ordinarily are prepared by site-specific mutagenesis of nucleotides in the DNA encoding a variant TNFSF protein, using cassette or PCR mutagenesis or other techniques well known in the art, to produce DNA encoding the variant, and thereafter expressing the DNA in recombinant cell culture as outlined above. However, variant TNFSF protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the variant TNFSF protein amino acid sequence. The variants typically exhibit the same qualitative biological activity as the naturally occurring analogue; although variants can also be selected which have modified characteristics.

While the site or region for introducing an amino acid sequence variation is predetermined, the mutation per se need not be predetermined. For example, in order to optimize the performance of a mutation at a given site, random mutagenesis may be conducted at the target codon or region and the expressed variant TNFSF proteins screened for the optimal combination of desired activity. Techniques for making substitution mutations at predetermined sites in DNA having a known sequence are well known, for example, M13 primer mutagenesis and PCR mutagenesis. Screening of the mutants is done using assays of variant TNFSF protein activities, or additional properties as outlined here (e.g. stability) for optimum characteristics.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule (e.g. conservative modifications). However, larger changes may be tolerated in certain circumstances (e.g. non-conservative modifications).

The variants typically exhibit the same qualitative biological activity and will elicit the same immune response as the original variant TNFSF protein, although variants also are selected to modify the characteristics of the variant TNFSF proteins as needed. Alternatively, the variant may be designed such that the biological activity of the variant TNFSF protein is altered. For example, glycosylation sites may be altered or removed. Similarly, the biological function may be altered; for example, in some instances it may be desirable to have more or less potent TNFSF activity.

The variant TNFSF proteins and nucleic acids of the invention can be made in a number of ways. Individual nucleic acids and proteins can be made as known in the art and outlined below. Alternatively, libraries of variant TNFSF proteins can be made for testing.

In a preferred embodiment, sets or libraries of variant TNFSF proteins may be generated in many ways known to those skilled in the art.

In a preferred embodiment, the different protein members of the variant TNFSF library may be chemically synthesized. This is particularly useful when the designed proteins are short, preferably less than 150 amino acids in length, with less than 100 amino acids being preferred, and less than 50 amino acids being particularly preferred, although as is known in the art, longer proteins may be made chemically or enzymatically. See for example Wilken et al, Curr. Opin. Biotechnol. 9:412-26 (1998), hereby expressly incorporated by reference.

In a preferred embodiment, particularly for longer proteins or proteins for which large samples are desired, the library sequences are used to create nucleic acids such as DNA which encode the member sequences and which may then be cloned into host cells, expressed and assayed, if desired. Thus, nucleic acids, and particularly DNA, may be made which encodes each member protein sequence. This is done using well-known procedures. The choice of codons, suitable expression vectors and suitable host cells will vary depending on a number of factors, and may be easily optimized as needed.

In a preferred embodiment, for example when libraries of variants are made for testing for antagonist activity and/or other desired activities as outlined herein, multiple PCR reactions with pooled oligonucleotides are done. In this embodiment, overlapping oligonucleotides are synthesized which correspond to the full-length gene. Again, these oligonucleotides may represent all of the different amino acids at each variant position or subsets. In a preferred embodiment, these oligonucleotides are pooled in equal proportions and multiple PCR reactions are performed to create full-length sequences containing the combinations of mutations defined by the library. In addition, this may be done using error-prone PCR methods.

In a preferred embodiment, the different oligonucleotides are added in relative amounts corresponding to a probability distribution table as described in U.S. Ser. No. 10/218,102. The multiple PCR reactions thus result in full-length sequences with the desired combinations of mutations in the desired proportions.

In a preferred embodiment, each overlapping oligonucleotide comprises only one position to be varied; in alternate embodiments, the variant positions are too close together to allow this and multiple variants per oligonucleotide are used to allow complete recombination of all the possibilities. That is, each oligo may contain the codon for a single position being mutated, or for more than one position being mutated. The multiple positions being mutated must be close in sequence to prevent the oligo length from being impractical. For multiple mutating positions on an oligonucleotide, particular combinations of mutations may be included or excluded in the library by including or excluding the oligonucleotide encoding that combination. For example, as discussed herein, there may be correlations between variable regions; that is, when position X is a certain residue, position Y must (or must not) be a particular residue. These sets of variable positions are sometimes referred to herein as a “cluster”. When the clusters are comprised of residues close together, and thus can reside on one oligonucleotide primer, the clusters can be set to the “good” correlations, and eliminate the bad combinations that may decrease the effectiveness of the library. However, if the residues of the cluster are far apart in sequence, and thus will reside on different oligonucleotides for synthesis, it may be desirable to either set the residues to the “good” correlation, or eliminate them as variable residues entirely. In an alternative embodiment, the library may be generated in several steps, so that the cluster mutations only appear together. This procedure, i.e. the procedure of identifying mutation clusters and either placing them on the same oligonucleotides or eliminating them from the library or library generation in several steps preserving clusters, can considerably enrich the experimental library with properly folded protein. Identification of clusters may be carried out by a number of ways, e.g. by using known pattern recognition methods, comparisons of frequencies of occurrence of mutations or by using energy analysis of the sequences to be experimentally generated (for example, if the energy of interaction is high, the positions are correlated). These correlations may be positional correlations (e.g. variable positions 1 and 2 always change together or never change together) or sequence correlations (e.g. if there is residue A at position 1, there is always residue B at position 2). See: Pattern discovery in Biomolecular Data: Tools, Techniques, and Applications; edited by Jason T. L. Wang, Bruce A. Shapiro, Dennis Shasha. New York: Oxford University, 1999; Andrews, Harry C. Introduction to mathematical techniques in pattern recognition; New York, Wiley-lnterscience [1972]; Applications of Pattern Recognition; Editor, K. S. Fu. Boca Raton, Fla. CRC Press, 1982; Genetic Algorithms for Pattern Recognition; edited by Sankar K. Pal, Paul P. Wang. Boca Raton: CRC Press, c1996; Pandya, Abhijit S., Pattern recognition with neural networks in C++/ Abhijit S. Pandya, Robert B. Macy. Boca Raton, Fla.: CRC Press, 1996; Handbook of pattern recognition & computer vision/edited by C. H. Chen, L. F. Pau, P. S. P. Wang. 2nd ed. Singapore; River Edge, N.J.: World Scientific, c1999; Friedman, Introduction to Pattern Recognition: Statistical, Structural, Neural, and Fuzzy Logic Approaches; River Edge, N.J.: World Scientific, c1999, Series title: Series in machine perception and artificial intelligence; vol. 32; all of which are expressly incorporated by reference. In addition, programs used to search for consensus motifs can be used as well.

Oligonucleotides with insertions or deletions of codons may be used to create a library expressing different length proteins. In particular computational sequence screening for insertions or deletions may result in secondary libraries defining different length proteins, which can be expressed by a library of pooled oligonucleotide of different lengths.

In another preferred embodiment, variant TNFSF proteins of the invention are created by shuffling the family (e.g. a set of variants); that is, some set of the top sequences (if a rank-ordered list is used) can be shuffled, either with or without error-prone PCR. “Shuffling” in this context means a recombination of related sequences, generally in a random way. It can include “shuffling” as defined and exemplified in U.S. Pat. Nos. 5,830,721; 5,811,238; 5,605,793; 5,837,458 and PCT US/19256, all of which are expressly incorporated by reference in their entirety. This set of sequences may also be an artificial set; for example, from a probability table (for example generated using SCMF) or a Monte Carlo set. Similarly, the “family” can be the top 10 and the bottom 10 sequences, the top 100 sequence, etc. This may also be done using error-prone PCR.

Thus, in a preferred embodiment, in silico shuffling is done using the computational methods described herein. That is, starting with two libraries or two sequences, random recombinations of the sequences may be generated and evaluated.

In a preferred embodiment variant TNFSF proteins are chimeras formed from two or more naturally occurring TNFSF proteins. In a particularly preferred embodiment, the chimeras are formed by joining one or more receptor contact region from one or more naturally occurring TNFSF proteins with the amino acid sequence of another naturally occurring TNFSF protein.

In a preferred embodiment, error-prone PCR is done to generate a library of variant TNFSF proteins. See U.S. Pat. Nos. 5,605,793, 5,811,238, and 5,830,721, all of which are hereby incorporated by reference. This may be done on the optimal sequence or on top members of the library, or some other artificial set or family. In this embodiment, the gene for the optimal sequence found in the computational screen of the primary library may be synthesized. Error-prone PCR is then performed on the optimal sequence gene in the presence of oligonucleotides that code for the mutations at the variant positions of the library (bias oligonucleotides). The addition of the oligonucleotides will create a bias favoring the incorporation of the mutations in the library. Alternatively, only oligonucleotides for certain mutations may be used to bias the library.

In a preferred embodiment, gene shuffling with error-prone PCR can be performed on the gene for the optimal sequence, in the presence of bias oligonucleotides, to create a DNA sequence library that reflects the proportion of the mutations found in the variant TNFSF library. The choice of the bias oligonucleotides can be done in a variety of ways; they can chosen on the basis of their frequency, i.e. oligonucleotides encoding high mutational frequency positions can be used; alternatively, oligonucleotides containing the most variable positions can be used, such that the diversity is increased; if the secondary library is ranked, some number of top scoring positions may be used to generate bias oligonucleotides; random positions may be chosen; a few top scoring and a few low scoring ones may be chosen; etc. What is important is to generate new sequences based on preferred variable positions and sequences.

In a preferred embodiment, PCR using a wild-type gene or other gene may be used, as is schematically depicted in the Figures. In this embodiment, a starting gene is used; generally, although this is not required, the gene is usually the wild-type gene. In some cases it may be the gene encoding the global optimized sequence, or any other sequence of the list, or a consensus sequence obtained e.g. from aligning homologous sequences from different organisms. In this embodiment, oligonucleotides are used that correspond to the variant positions and contain the different amino acids of the library. PCR is done using PCR primers at the termini, as is known in the art. This provides two benefits. First, this generally requires fewer oligonucleotides. and may result in fewer errors. Second, it has experimental advantages in that if the wild-type gene is used, it need not be synthesized.

Using the nucleic acids of the present invention which encode a variant TNFSF protein, a variety of expression vectors are made. The expression vectors may be either self-replicating extrachromosomal vectors or vectors which integrate into a host genome. Generally, these expression vectors include transcriptional and translational regulatory nucleic acid operably linked to the nucleic acid encoding the variant TNFSF protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation.

In a preferred embodiment, when the endogenous secretory sequence leads to a low level of secretion of the naturally occurring protein or of the variant TNFSF protein, a replacement of the naturally occurring secretory leader sequence is desired. In this embodiment, an unrelated secretory leader sequence is operably linked to a variant TNFSF encoding nucleic acid leading to increased protein secretion. Thus, any secretory leader sequence resulting in enhanced secretion of the variant TNFSF protein, when compared to the secretion of TNFSF and its secretory sequence, is desired. Suitable secretory leader sequences that lead to the secretion of a protein are known in the art.

In another preferred embodiment, a secretory leader sequence of a naturally occurring protein or a protein is removed by techniques known in the art and subsequent expression results in intracellular accumulation of the recombinant protein.

Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express the fusion protein; for example, transcriptional and translational regulatory nucleic acid sequences from Bacillus are preferably used to express the fusion protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention. In a preferred embodiment, the promoters are strong promoters, allowing high expression in cells, particularly mammalian cells, such as the CMV promoter, particularly in combination with a Tet regulatory element.

In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used.

A preferred expression vector system is a retroviral vector system such as is generally described in PCT/US97/01019 and PCT/US97/01048, both of which are hereby expressly incorporated by reference.

In a preferred embodiment, the expression vector comprises the components described above and a gene encoding a variant TNFSF protein. As will be appreciated by those in the art, all combinations are possible and accordingly, as used herein, the combination of components, comprised by one or more vectors, which may be retroviral or not, is referred to herein as a “vector composition”.

The variant TNFSF nucleic acids are introduced into the cells either alone or in combination with an expression vector. By “introduced into ” or grammatical equivalents herein is meant that the nucleic acids enter the cells in a manner suitable for subsequent expression of the nucleic acid. The method of introduction is largely dictated by the targeted cell type, discussed below. Exemplary methods include CaPO₄ precipitation, liposome fusion, lipofectin®, electroporation, viral infection, etc. The variant TNFSF nucleic acids may stably integrate into the genome of the host cell (for example, with retroviral introduction, outlined below), or may exist either transiently or stably in the cytoplasm (i.e. through the use of traditional plasmids, utilizing standard regulatory sequences, selection markers, etc.).

The variant TNFSF proteins of the present invention are produced by culturing a host cell transformed with an expression vector containing nucleic acid encoding a variant TNFSF protein, under the appropriate conditions to induce or cause expression of the variant TNFSF protein. The conditions appropriate for variant TNFSF protein expression wili vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fungi, and insect and animal cells, including mammalian cells. Of particular interest are Drosophila melangaster cells, Saccharomyces cerevisiae and other yeasts, E. coli, Bacillus subtilis, SF9 cells, C129 cells, 293 cells, Neurospora, BHK, CHO, COS, Pichia pastoris, etc.

In a preferred embodiment, the variant TNFSF proteins are expressed in mammalian cells. Mammalian expression systems are also known in the art, and include retroviral systems. A mammalian promoter is any DNA sequence capable of binding mammalian RNA polymerase and initiating the downstream (3′) transcription of a coding sequence for the fusion protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei. As outlined herein, a particularly preferred method utilizes retroviral infection, as outlined in PCT US97/01019, incorporated by reference.

As will be appreciated by those in the art, the type of mammalian cells used in the present invention can vary widely. Basically, any mammalian cells may be used, with mouse, rat, primate and human cells being particularly preferred, although as will be appreciated by those in the art, modifications of the system by pseudotyping allows all eukaryotic cells to be used, preferably higher eukaryotes. As is more fully described below, a screen will be set up such that the cells exhibit a selectable phenotype in the presence of a bioactive peptide. As is more fully described below, cell types implicated in a wide variety of disease conditions are particularly useful, so long as a suitable screen may be designed to allow the selection of cells that exhibit an altered phenotype as a consequence of the presence of a peptide within the cell.

Accordingly, suitable cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoietic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, Cos, etc. See the ATCC cell line catalog, hereby expressly incorporated by reference.

In one embodiment, the cells may be additionally genetically engineered, that is, contain exogenous nucleic acid other than the variant TNFSF nucleic acid.

In a preferred embodiment, the variant TNFSF proteins are expressed in bacterial systems. Bacterial expression systems are well known in the art.

A suitable bacterial promoter is any nucleic acid sequence capable of binding bacterial RNA polymerase and initiating the downstream (3′) transcription of the coding sequence of the variant TNFSF protein into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter may include naturally occurring promoters of non-bacterial origin that have the ability to bind bacterial RNA polymerase and initiate transcription.

In addition to a functioning promoter sequence, an efficient ribosome binding site is desirable. In E. coli, the ribosome binding site is called the Shine-Delgarno (SD) sequence and includes an initiation codon and a sequence 3-9 nucleotides in length located 3-11 nucleotides upstream of the initiation codon.

The expression vector may also include a signal peptide sequence that provides for secretion of the variant TNFSF protein in bacteria. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria). For expression in bacteria, usually bacterial secretory leader sequences, operably linked to a variant TNFSF encoding nucleic acid, are preferred.

The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways.

These components are assembled into expression vectors. Expression vectors for bacteria are well known in the art, and include vectors for Bacillus subtilis, E. coli, Streptococcus cremoris, and Streptococcus lividans, among others.

The bacterial expression vectors are transformed into bacterial host cells using techniques well known in the art, such as calcium chloride treatment, electroporation, and others.

In one embodiment, variant TNFSF proteins are produced in insect cells. Expression vectors for the transformation of insect cells, and in particular, baculovirus-based expression vectors, are well known in the art.

In a preferred embodiment, variant TNFSF protein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. Preferred promoter sequences for expression in yeast include the inducible GAL1, 10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include ADE2, HIS4, LEU2, TRP1, and ALG7, which confers resistance to tunicamycin; the neomycin phosphotransferase gene, which confers resistance to G418; and the CUP1 gene, which allows yeast to grow in the presence of copper ions.

In a preferred embodiment, modified TNFSF variants are covalently coupled to at least one additional TNFSF variant via a linker to improve the dominant negative action of the modified domains. A number of strategies may be used to covalently link modified receptor domains together. These include, but are not limited to, linkers, such as polypeptide linkages between N- and C-termini of two domains, linkage via a disulfide bond between monomers, and linkage via chemical cross-linking reagents. Alternatively, the N- and C- termini may be covalently joined by deletion of portions of the N- and/or C-termini and linking the remaining fragments via a linker or linking the fragments directly.

By “linker”, “linker sequence”, “spacer”, “tethering sequence” or grammatical equivalents thereof, herein is meant a molecule or group of molecules (such as a monomer or polymer) that connects two molecules and often serves to place the two molecules in a preferred configuration. In one aspect of this embodiment, the linker is a peptide bond. Choosing a suitable linker for a specific case where two polypeptide chains are to be connected depends on various parameters, e.g., the nature of the two polypeptide chains (e.g., whether they naturally oligomerize (e.g., form a dimer or not), the distance between the N- and the C-termini to be connected if known from three-dimensional structure determination, and/or the stability of the linker towards proteolysis and oxidation. Furthermore, the linker may contain amino acid residues that provide f:exibility. Thus, the linker peptide may predominantly include the following amino acid residues: Gly, Ser, Ala, or Thr. These linked TNFSF proteins have constrained hydrodynamic properties, that is, they form constitutive dimers) and thus efficiently interact with other naturally occurring TNFSF proteins to form a dominant negative heterotrimer.

The linker peptide should have a length that is adequate to link two TNFSF variant monomers in such a way that they assume the correct conformation relative to one another so that they retain the desired activity as antagonists of the native TNFSF protein. Suitable lengths for this purpose include at least one and not more than 30 amino acid residues. Preferably, the linker is from about 1 to 30 amino acids in length, with linkers of 1, 2, 3, 4, 5, 6, 7, 8, 9, 10, 11, 12, 13, 14, 15, 16, 17, 18 19 and 20 amino acids in length being preferred. See also WO 01/25277, incorporated herein by reference in its entirety.

In addition, the amino acid residues selected for inclusion in the linker peptide should exhibit properties that do not interfere significantly with the activity of the polypeptide. Thus, the linker peptide on the whole should not exhibit a charge that would be inconsistent with the activity of the polypeptide, or interfere with internal folding, or form bonds or other interactions with amino acid residues in one or more of the monomers that would seriously impede the binding of receptor monomer domains.

Useful linkers include glycine-serine polymers (including, for example, (GS)n, (GSGGS)n (GGGGS)n and (GGGS)n, where n is an integer of at least one), glycine-alanine polymers, alanine-serine polymers, and other flexible linkers such as the tether for the shaker potassium channel, and a large variety of other flexible linkers, as will be appreciated by those in the art; Glycine-serine polymers are preferred since both of these amino acids are relatively unstructured, and therefore may be able to serve as a neutral tether between components. Secondly, serine is hydrophilic and therefore able to solubilize what could be a globular glycine chain. Third, similar chains have been shown to be effective in joining subunits of recombinant proteins such as single chain antibodies.

Suitable linkers may also be identified by screening databases of known three-dimensional structures for naturally occurring motifs that can bridge the gap between two polypeptide chains. Another way of obtaining a suitable linker is by optimizing a simple linker, e.g., (Gly4Ser)n, through random mutagenesis. Alternatively, once a suitable polypeptide linker is defined, additional linker polypeptides can be created by application of PDATM technology to select amino acids that more optimally interact with the domains being linked. Other types of linkers that may be used in the present invention include artificial polypeptide linkers and inteins. In another preferred embodiment, disulfide bonds are designed to link the two receptor monomers at inter-monomer contact sites. In one aspect of this embodiment the two receptors are linked at distances <5 Angstroms. In addition, the variant TNFSF polypeptides of the invention may be further fused to other proteins, if desired, for example to increase expression or stabilize the protein.

In one embodiment, the variant TNFSF nucleic acids, proteins and antibodies of the invention are labeled with a label other than the scaffold. By “labeled” herein is meant that a compound has at least one element, isotope or chemical compound attached to enable the detection of the compound. In general, labels fall into three classes: a) isotopic labels, which may be radioactive or heavy isotopes; b) immune labels, which may be antibodies or antigens; and c) colored or fluorescent dyes. The labels may be incorporated into the compound at any position.

Once made, the variant TNFSF proteins may be modified. Covalent and non-covalent modifications of the protein are included within the scope of the present invention. Such modifications may be introduced into a variant TNFSF polypeptide by reacting targeted amino acid residues of the polypeptide with an organic derivatizing agent that is capable of reacting with selected side chains or terminal residues.

One type of covalent modification includes reacting targeted amino acid residues of a variant TNFSF polypeptide with an organic derivatizing agent that is capable of reacting with selected side chains or the N-or C-terminal residues of a variant TNFSF polypeptide. Derivatization with bifunctional agents is useful, for instance, for cross linking a variant TNFSF protein to a water-insoluble support matrix or surface for use in the method for purifying anti-variant TNFSF antibodies or screening assays, as is more fully described below. Commonly used cross linking agents include, e.g., 1,1-bis(diazoacetyi)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidyl-propionate), bifunctional maleimides such as bis-N-maleimido-1,8-octane and agents such as methyl-3-[(p-azidophenyl)dithio] propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the “-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Another type of covalent modification of the variant TNFSF polypeptide included within the scope of this invention comprises altering the native glycosylation pattern of the polypeptide. “Altering the native glycosylation pattern” is intended for purposes herein to mean deleting one or more carbohydrate moieties found in native sequence variant TNFSF polypeptide, and/or adding one or more glycosylation sites that are not present in the native sequence variant TNFSF polypeptide.

Addition of glycosylation sites to variant TNFSF polypeptides may be accomplished by altering the amino acid sequence thereof. The alteration may be made, for example, by the addition of, or substitution by, one or more serine or threonine residues to the native sequence or variant TNFSF polypeptide (for O-linked glycosylation sites). The variant TNFSF amino acid sequence may optionally be altered through changes at the DNA level, particularly by mutating the DNA encoding the variant TNFSF polypeptide at preselected bases such that codons are generated that will translate into the desired amino acids.

Addition of N-linked glycosylation sites to variant TNFSF polypeptides may be accomplished by altering the amino acid sequence thereof. The alteration may be made, for example, by the addition of, or substitution by, one or more asparagine residues to the native sequence or variant TNFSF polypeptide. The modification may be made for example by the incorporation of a canonical N-linked glycosylation site, including but not limited to, N—X—Y, where X is any amino acid except for proline and Y is preferably threonine, serine or cysteine. Another means of increasing the number of carbohydrate moieties on the variant TNFSF polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the variant TNFSF polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo-and exo-glycosidases as described by Thotakura et al., Meth. Enzymol., 138:350 (1987).

Such derivatized moieties may improve the solubility, absorption, and permeability across the blood brain barrier biological half-life, and the like. Such moieties or modifications of variant TNFSF polypeptides may alternatively eliminate or attenuate any possible undesirable side effect of the protein and the like. Moieties capable of mediating such effects are disclosed, for example, in Remington's Pharmaceutical Sciences, 16th ed., Mack Publishing Co., Easton, Pa. (1980).

Another type of covalent modification of variant TNFSF comprises linking the variant TNFSF polypeptide to one of a variety of nonproteinaceous polymers, e.g., polyethylene glycol (“PEG”), polypropylene glycol, or polyoxyalkylenes, in the manner set forth in U.S. Pat. Nos. 4,640,835; 4,496,689; 4,301,144; 4,670,417; 4,791,192; 4,179,337; 5,183,550. These nonproteinaceous polymers may also be used to enhance the variant TNFSF's ability to disrupt receptor binding, and/or in vivo stability.

In another preferred embodiment, cysteines are designed into variant or wild-type TNFSF in order to incorporate (a) labeling sites for characterization and (b) incorporate PEGylation sites. For example, labels that may be used are well known in the art and include but are not limited to biotin, tag and fluorescent labels (e.g. fluorescein). These labels may be used in various assays as are also well known in the art to achieve characterization. A variety of coupling chemistries may be used to achieve PEGylation, as is well known in the art. Examples include but are not limited to, the technologies of Shearwater and Enzon, which allow modification at primary amines, including but not limited to, lysine groups and the N-terminus. See, Kinstler et al, Advanced Drug Deliveries Reviews, 54, 477-485 (2002) and MJ Roberts et al, Advanced Drug Delivery Reviews, 54, 459-476 (2002), both hereby incorporated by reference.

Other modifications may be made to the variant TNFSF proteins of the present invention, including modifications to the protein that enhance stability, dosage administration (e.g., amphiphilic polymers, see WO 0141812A2, commercially available from Nobex Corporation), clearance (e.g., PEG, aliphatic moieties that effect binding to HSA), and the like.

Optimal sites for modification can be chosen using a variety of criteria, including but not limited to, visual inspection, structural analysis, sequence analysis and molecular simulation. Individual residues may be analyzed to identify mutational sites that will not disrupt the monomer structure. Then the distance from each side chain of a monomer to another subunit may be calculated to ensure that chemical modification will not disrupt oligomerization. It is possible that receptor binding disruption may occur and may be beneficial to the activity of the TNFSF variants of this invention.

In another preferred embodiment, portions of either the N- or C- termini of the wild-type TNFSF monomer are deleted while still allowing the TNFSF molecule to fold properly. In addition, these modified TNFSF proteins would substantially lack receptor binding and/or activation, and could optionally interact with other wild-type TNFSF molecules or modified TNFSF proteins to form trimers (or other oligomers) as described above.

More specifically, removal or deletion of from about 1 to about 55 amino acids from either the N or C termini, or both, are preferred. A more preferred embodiment includes deletions of N-termini beyond residue 10 and more preferably, deletion of the first 47 N-terminal amino acids. The deletion of C-terminal leucine is an alternative embodiment.

In another preferred embodiment, the wild-type TNFSF or variants generated by the invention may be circularly permuted. All natural proteins have an amino acid sequence beginning with an N-terminus and ending with a C-terminus. The N- and C-termini may be joined to create a cyclized or circularly permutated TNFSF proteins while retaining or improving biological properties (e.g., such as enhanced stability and activity) as compared to the wild-type protein. In the case of a TNFSF protein, a novel set of N- and C-termini are created at amino acid positions normally internal to the protein's primary structure, and the original N- and C-termini are joined via a peptide linker consisting of from 0 to 30 amino acids in length (in some cases, some of the amino acids located near the original termini are removed to accommodate the linker design). In a preferred embodiment, the novel N- and C-termini are located in a non-regular secondary structural element, such as a loop or turn, such that the stability and activity of the novel protein are similar to those of the original protein. The circularly permuted TNFSF protein may be further PEGylated or glycosylated. In a further preferred embodiment PDA™ technology may be used to further optimize the TNFSF variant, particularly in the regions created by circular permutation. These include the novel N- and C-termini, as well as the original termini and linker peptide.

Various techniques may be used to permutate proteins. See U.S. Pat. No. 5,981,200; Maki K, Iwakura M., Seikagaku. 2001 January; 73(1): 42-6; Pan T., Methods Enzymol. 2000; 317:313-30; Heinemann U, Hahn M., Prog Biophys Mol Biol. 1995; 64(2-3): 121-43; Harris M E, Pace N R, Mol Biol Rep. 1995-96; 22(2-3):115-23; Pan T, Uhlenbeck O C., 1993 Mar. 30; 125(2): 111-4; Nardulli A M, Shapiro D J. 1993 Winter; 3(4):247-55, EP 1098257 A2; WO 02/22149; WO 01/51629; WO 99/51632; Hennecke, et al., 1999, J. Mol. Biol., 286, 1197-1215; Goldenberg et al J. Mol. Biol 165, 407-413 (1983); Luger et al, Science, 243, 206-210 (1989); and Zhang et al., Protein Sci 5, 1290-1300 (1996); all hereby incorporated by reference.

In addition, a completely cyclic TNFSF may be generated, wherein the protein contains no termini. This is accomplished utilizing intein technology. Thus, peptides can be cyclized and in particular inteins may be utilized to accomplish the cyclization.

Cyclization and circular permutation may be used to generate the dominant-negative activity of the TNFSF proteins of the present invention.

Variant TNFSF polypeptides of the present invention may also be modified in a way to form chimeric molecules comprising a variant TNFSF polypeptide fused to another, heterologous polypeptide or amino acid sequence. In one embodiment, such a chimeric molecule comprises a fusion of a variant TNFSF polypeptide with a tag polypeptide that provides an epitope to which an anti-tag antibody can selectively bind. The epitope tag is generally placed at the amino-or carboxyl-terminus of the variant TNFSF polypeptide. The presence of such epitope-tagged forms of a variant TNFSF polypeptide can be detected using an antibody against the tag polypeptide. Also, provision of the epitope tag enables the variant TNFSF polypeptide to be readily purified by affinity purification using an anti-tag antibody or another type of affinity matrix that binds to the epitope tag. In an alternative embodiment, the chimeric molecule may comprise a fusion of a variant TNFSF polypeptide with an immunoglobulin or a particular region of an immunoglobulin. For a bivalent form of the chimeric molecule, such a fusion could be to the Fc or Fab region of an IgG molecule. Other fusion entities include human serum albumin (HSA), hydrophilic peptides, fatty acid molecules, labels, etc.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol. 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6): 547-553 (1990)]. Other tag polypeptides include the Flag-peptide [Hopp et al., BioTechnology 6:1204-1210 (1988)]; the KT3 epitope peptide [Martin et al., Science 255:192-194 (1992)]; tubulin epitope peptide [Skinner et al., J. Biol. Chem. 266:15163-15166 (1991)]; and the T7 gene 10 protein peptide tag [Lutz-Freyermuth et al., Proc. Natl. Acad. Sci. U.S.A. 87:6393-6397 (1 990)].

In a preferred embodiment, the variant TNFSF protein is purified or isolated after expression. Variant TNFSF proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing.

For example, the variant TNFSF protein may be purified using a standard anti-library antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the variant TNFSF protein. In some instances no purification will be necessary.

In a preferred embodiment, the variant TNFSF proteins of the present invention are produced and purified separated from the naturally occurring TNFSF proteins that are antagonized. That is, variant monomers are made and introduced to the wild-type proteins, either in monomeric or oligomeric form. For example, a preferred method involves the administration of variant monomers to a patient, whereby the variant monomers “exchange” into the homo-oligomers (generally trimers), to produce mixed oligomers that result in reduced receptor activation. In alternative embodiments, variant oligomers may be administered that then exchange. However, in some embodiments, such as in gene therapy applications, the variant TNFSF proteins may be produced substantially simulataneously with the naturally occurring TNFSF targets.

Once made, the variant TNFSF proteins and nucleic acids of the invention find use in a number of applications. In a preferred embodiment, the variant TNFSF proteins are administered to a patient to treat a TNFSF related disorder.

By “TNFSF related disorder” or “TNFSF responsive disorder” or “condition” herein is meant a disorder that may be ameliorated by the administration of a pharmaceutical composition comprising a variant TNFSF protein, including, but not limited to, autoimmune, inflammatory and immunological disorders. The variant TNFSF proteins are major effectors in the pathogenesis of immune-regulated diseases.

In a preferred embodiment, the variant TNFSF protein is used to treat for example, congestive heart failure (CHF), vasculitis, rosecea, acne, excema, myocarditis and other conditions of the myocardium, systemic lupus erythematosus, diabetes, spondylopathies, synovial fibroblasts, and bone marrow stroma; bone loss; Paget's disease, osteoclastoma; multiple myeloma; breast cancer; disuse osteopenia; malnutrition, periodontal disease, Gaucher's disease, Langerhans' cell histiocytosis, spinal cord injury, acute septic arthritis, osteomalacia, Cushing's syndrome, monoostotic fibrous dysplasia, polyostotic fibrous dysplasia, periodontal reconstruction, and bone fractures; sarcoidosis; multiple myeloma; osteolytic bone cancers, breast cancer, lung cancer, kidney cancer and rectal cancer; bone metastasis, bone pain management, and humoral malignant hypercalcemia, ankylosing spondylitisa and other spondyloarthropathies; transplantation rejection, viral infections, hematologic neoplasisas and neoplastic-like conditions for example, Hodgkin's lymphoma; non-Hodgkin's lymphomas (Burkitt's lymphoma, small lymphocytic lymphoma/chronic lymphocytic leukemia, mycosis fungoides, mantle cell lymphoma, follicular lymphoma, diffuse large B-cell lymphoma, marginal zone lymphoma, hairy cell leukemia and lymphoplasmacytic leukemia), tumors of lymphocyte precursor cells, including B-cell acute lymphoblastic leukemia/lymphoma, and T-cell acute lymphoblastic leukemia/lymphoma, thymoma, tumors of the mature T and NK cells, including peripheral T-cell leukemias, adult T-cell leukemia/T-cell lymphomas and large granular lymphocytic leukemia, Langerhans cell histocytosis, myeloid neoplasias such as acute myelogenous leukemias, including AML with maturation, AML without differentiation, acute promyelocytic leukemia, acute myelomonocytic leukemia, and acute monocytic leukemias, myelodysplastic syndromes, and chronic myeloproliferative disorders, including chronic myelogenous leukemia, tumors of the central nervous system, e.g., brain tumors (glioma, neuroblastoma, astrocytoma, medulloblastoma, ependymoma, and retinoblastoma), solid tumors (nasopharyngeal cancer, basal cell carcinoma, pancreatic cancer, cancer of the bile duct, Kaposi's sarcoma, testicular cancer, uterine, vaginal or cervical cancers, ovarian cancer, primary liver cancer or endometrial cancer, and tumors of the vascular system (angiosarcoma and hemagiopericytoma), osteoporosis, hepatitis, HIV, AIDS, spondyloarthritis, rheumatoid arthritis, inflammatory bowel diseases (IBD), sepsis and septic shock, Crohn's Disease, psoriasis, schleraderma, graft versus host disease (GVHD), allogenic islet graft rejection, hematologic malignancies, such as multiple myeloma (MM), myelodysplastic syndrome (MDS) and acute myelogenous leukemia (AML), cancer and the inflammation associated with tumors, peripheral nerve injury or demyelinating diseases.

By “therapeutically effective dose” herein is meant a dose that produces the effects for which it is administered. The exact dose will depend on the purpose of the treatment, and will be ascertainable by one skilled in the art using known techniques. In a preferred embodiment, dosages of about 0.01 to about 50 μg/kg are used, administered either intravenously or subcutaneously. As is known in the art, adjustments for variant TNFSF protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

A “patient” for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human.

The term “treatment” in the instant invention is meant to include therapeutic treatment, as well as prophylactic, or suppressive measures for the disease or disorder. Thus, for example, successful administration of a variant TNFSF protein prior to onset of the disease results in “treatment” of the disease. As another example, successful administration of a variant TNFSF protein after clinical manifestation of the disease to combat the symptoms of the disease comprises “treatment” of the disease. “Treatment” also encompasses administration of a variant TNFSF protein after the appearance of the disease in order to eradicate the disease. Successful administration of an agent after onset and after clinical symptoms have developed, with possible abatement of clinical symptoms and perhaps amelioration of the disease, comprises “treatment” of the disease.

Those “in need of treatment” include mammals already having the disease or disorder, as well as those prone to having the disease or disorder, including those in which the disease or disorder is to be prevented.

In another embodiment, a therapeutically effective dose of a variant TNFSF protein, a variant TNFSF gene, or a variant TNFSF antibody is administered to a patient having a disease involving inappropriate expression of a TNFSF protein. A “disease involving inappropriate expression of a TNFSF protein” within the scope of the present invention is meant to include diseases or disorders characterized by aberrant TNFSF proteins, either by alterations in the amount of TNFSF protein present or due to the presence of mutant TNFSF protein. An overabundance may be due to any cause, including, but not limited to, overexpression at the molecular level, prolonged or accumulated appearance at the site of action, or increased activity of TNFSF protein relative to normal. Included within this definition are diseases or disorders characterized by a reduction of TNFSF protein. This reduction may be due to any cause, including, but not limited to, reduced expression at the molecular level, shortened or reduced appearance at the site of action, mutant forms of TNFSF protein, or decreased activity of TNFSF protein relative to normal. Such an overabundance or reduction of TNFSF protein can be measured relative to normal expression, appearance, or activity of TNFSF protein according to, but not limited to, the assays described and referenced herein.

The administration of the variant TNFSF proteins of the present invention, preferably in the form of a sterile aqueous solution, may be done in a variety of ways, including, but not limited to, orally, subcutaneously, intravenously, intranasally, intraotically, transdermally, topically (e.g., gels, salves, lotions, creams, etc.), intraperitoneally, intramuscularly, intrapulmonary (e.g., AERx® inhalable technology commercially available from Aradigm or Inhance™ pulmonary delivery system commercially available from Inhale Therapeutics), vaginally, rectally, or intraocularly. In some instances, for example, in the treatment of wounds, inflammation, etc., the variant TNFSF protein may be directly applied as a solution or spray. Depending upon the manner of introduction, the pharmaceutical composition may be formulated in a variety of ways.

Also, sustained release or controlled release formulations may be used for the compositions of the present invention. For example, ProLease® (commercially available from Alkermes) a microsphere-based delivery system composed of the desired bioactive molecule incorporated into a matrix of poly-DL-lactide-co-glycolide (PLG) and other pharmaceutically compatible polymeric matrices may be used to create sustained release formulations.

The concentration of the therapeutically active variant TNFSF protein in the formulation may vary from about 0.1 to 100 weight %. In another preferred embodiment, the concentration of the variant TNFSF protein is in the range of 0.003 to 1.0 molar, with dosages from 0.03, 0.05, 0.1, 0.2, and 0.3 millimoles per kilogram of body weight being preferred.

The pharmaceutical compositions of the present invention comprise a variant TNFSF protein in a form suitable for administration to a patient. In the preferred embodiment, the pharmaceutical compositions are in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations.

In a further embodiment, the variant TNFSF proteins are added in a micellular formulation; see U.S. Pat. No. 5,833,948, hereby expressly incorporated by reference in its entirety.

Combinations of pharmaceutical compositions may be administered. Moreover, the compositions may be administered in combination with other therapeutics.

In one embodiment provided herein, antibodies, including but not limited to monoclonal and polyclonal antibodies, are raised against variant TNFSF proteins using methods known in the art. In a preferred embodiment, these anti-variant TNFSF antibodies are used for immunotherapy. Thus, methods of immunotherapy are provided. By “immunotherapy” is meant treatment of a TNFSF related disorders with an antibody raised against a variant TNFSF protein. As used herein, immunotherapy can be passive or active. Passive immunotherapy, as defined herein, is the passive transfer of antibody to a recipient (patient). Active immunization is the induction of antibody and/or T-cell responses in a recipient (patient). Induction of an immune response can be the consequence of providing the recipient with a variant TNFSF protein antigen to which antibodies are raised. As appreciated by one of ordinary skill in the art, the variant TNFSF protein antigen may be provided by injecting a variant TNFSF polypeptide against which antibodies are desired to be raised into a recipient, or contacting the recipient with a variant TNFSF protein encoding nucleic acid, capable of expressing the variant TNFSF protein antigen, under conditions for expression of the variant TNFSF protein antigen.

In another preferred embodiment, a therapeutic compound is conjugated to an antibody, preferably an anti-variant TNFSF protein antibody. The therapeutic compound may be a cytotoxic agent. In this method, targeting the cytotoxic agent to tumor tissue or cells, results in a reduction in the number of afflicted cells, thereby reducing symptoms associated with cancer, and variant TNFSF protein related disorders. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin and the like. Cytotoxic agents also include radiochemicals made by conjugating radioisotopes to antibodies raised against cell cycle proteins, or binding of a radionuclide to a chelating agent that has been covalently attached to the antibody.

In a preferred embodiment, variant TNFSF proteins are administered as therapeutic agents, and can be formulated as outlined above. Similarly, variant TNFSF genes (including both the full-length sequence, partial sequences, or regulatory sequences of the variant TNFSF coding regions) may be administered in gene therapy applications, as is known in the art. These variant TNFSF genes can include antisense applications, either as gene therapy (i.e. for incorporation into the genome) or as antisense compositions, as will be appreciated by those in the art.

In a preferred embodiment, the nucleic acid encoding the variant TNFSF proteins may also be used in gene therapy. In gene therapy applications, genes are introduced into cells in order to achieve in vivo synthesis of a therapeutically effective genetic product, for example for replacement of a defective gene. “Gene therapy” includes both conventional gene therapy, where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. [Zamecnik et al., Proc. Natl. Acad. Sci. U.S.A. 83:4143-4146 (1986)]. The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection [Dzau et al., Trends in Biotechnology 11:205-210 (1993)]. In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. U.S.A. 87:3410-3414 (1990). For review of gene marking and gene therapy protocols see Anderson et al., Science 256:808-813 (1992).

In another embodiment, variant TNFSF genes are administered as DNA vaccines, either single genes or combinations of variant TNFSF genes. Naked DNA vaccines are generally known in the art. Brower, Nature Biotechnology, 16:1304-1305 (1998). Methods for the use of genes as DNA vaccines are well known to one of ordinary skill in the art, and include placing a variant TNFSF gene or portion of a variant TNFSF gene under the control of a promoter for expression in a patient in need of treatment.

The variant TNFSF gene used for DNA vaccines can encode full-length variant TNFSF proteins, but more preferably encodes portions of the variant TNFSF proteins including peptides derived from the variant TNFSF protein. In a preferred embodiment a patient is immunized with a DNA vaccine comprising a plurality of nucleotide sequences derived from a variant TNFSF gene. Similarly, it is possible to immunize a patient with a plurality of variant TNFSF genes or portions thereof as defined herein. Without being bound by theory, expression of the polypeptide encoded by the DNA vaccine, cytotoxic T-cells, helper T-cells and antibodies are induced which recognize and destroy or eliminate cells expressing TNFSF proteins.

In a preferred embodiment, the DNA vaccines include a gene encoding an adjuvant molecule with the DNA vaccine. Such adjuvant molecules include cytokines that increase the immunogenic response to the variant TNFSF polypeptide encoded by the DNA vaccine. Additional or alternative adjuvants are known to those of ordinary skill in the art and find use in the invention.

All references cited herein, including patents, patent applications (provisional, utility and PCT), and publications are incorporated by reference in their entirety.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes.

### EXAMPLES

TNF-alpha Library Expression, Purification, and Activity Assavs for TNF-alpha Variants

Methods:

1) Overnight culture preparation:

Competent Tuner(DE3)pLysS cells in 96 well-PCR plates were transformed with 1 ul of TNF-alpha library DNAs and spread on LB agar plates with 34 mg/ml chloramphenicol and 100 mg/ml ampicillin. After an overnight growth at 37 degrees C., a colony was picked from each plate in 1.5 ml of CG media with 34 mg/ml chloramphenicol and 100 mg/ml ampicillin kept in 96 deep well block. The block was shaken at 250 rpm at 37 degrees C. overnight.

2) Expression:

Colonies were picked from the plate into 5 ml CG media (34 mg/ml chloramphenicol and 100 mg/ml ampicillin) in 24-well block and grown at 37 degrees C. at 250 rpm until OD600 0.6 were reached, at which time IPTG was added to each well to 1 mM concentration. The culture was grown 4 extra hours.

3) Lysis:

The 24-well block was centrifuged at 3000 rpm for 10 minutes. The pellets were resuspended in 700 ul of lysis buffer (50 mM NaH2PO4, 300 mM NaCI, 10 mM imidazole). After freezing at −80 degrees C. for 20 minutes and thawing at 37 degrees C. twice, MgCl2 was added to 10 mM, and DNase 1 to 75 mg/ml. The mixture was incubated at 37 degrees C. for 30 minutes.

4) Ni NTA column purification:

Purification was carried out following Qiagen Ni NTA spin column purification protocol for native condition. The purified protein was dialyzed against 1×PBS for 1 hour at 4 degrees C. four times. Dialyzed protein was filter sterilized, using Millipore multiscreenGV filter plate to allow the addition of protein to the sterile mammalian cell culture assay later on.

5) Quantification:

Purified protein was quantified by SDS PAGE, followed by Coomassie stain, and by Kodak™ digital image densitometry.

TNF-Alpha Antagonist Activity

A) Materials and Methods: Plated cells for the assay: WEHI plated at 2.5×105 cells/ml (50 μl/well) Prepared Assay Media as follows: 1×Assay Medium, 40 ml complete RPMI medium, 80 μl Actinomycin D (2 μg/ml final concentration). Prepared assay medium+wild-type TNF-alpha (Wild-type TNF-alpha is 1.1 mg/ml-1 μg/ml: 1:1000; 1 μl of the stock in 1 ml of RPMI-20 ng/ml: 1:50 of the 1 μg/ml; 800 μl in 40 ml of assay medium Dilution of TNF-alpha variants was done as shown below:

| | Assay | | | | |
|---|---|---|---|---|---|
| | medium (μl) | | | | |
| | with 20 | Concentration | | Final conc. | |
| | ng/ml | in | | of TNF-a | |
| Stock (μl) | of wt TNF-a | dilution plate | | in cells | |
| K112D: 59 μl | 941 | 100,000 | ng/ml | 50,000 | ng/ml |
| Y115T: 77 μl | 923 | | | | |
| D143K: 32 μl | 968 | | | | |
| D143R: 34 μl | 966 | | | | |
| Y115I: 63 μl | 937 | | | | |
| D143E: 40 μl | 960 | | | | |
| A145R: 50 μl | 950 | | | | |
| A145K: 50 μl | 950 | | | | |
| A145E: 26 μl | 974 | | | | |
| E146K: 40 μl | 960 | | | | |
| E146R: 56 μl | 944 | | | | |
| 500 μl of 100,000 | 500 | 50,000 | ng/ml | 25,000 | ng/ml |
| ng/ml | | | | | |
| 500 μl of 50,000 ng/ml | 500 | 25,000 | ng/ml | 12,500 | ng/ml |
| 400 μl of 25,000 ng/ml | 600 | 10,000 | ng/ml | 5000 | ng/ml |
| 500 μl of 10,000 ng/ml | 500 | 5,000 | ng/ml | 2,500 | ng/ml |
| 200 μl of 5000 ng/ml | 800 | 1000 | ng/ml | 500 | ng/mL |
| 500 μl of 1000 ng/ml | 500 | 500 | ng/ml | 50 | ng/mL |
| 500 μl of the 500 | 500 | 250 | ng/ml | 125 | ng/mL |
| ng/ml | | | | | |
| 400 μl of 250 ng/ml | 600 | 100 | ng/ml | 50 | ng/mL |
| 100 μl of 100 ng/ml | 900 | 10 | ng/ml | 5 | ng/mL |
| 100 μl of 10 ng/ml | 900 | 1 | ng/ml | 0.5 | ng/mL |
| 0 | 0 | 0 | | 0 | |

5) Dilutions for Inhibition Assay:

Stocks to dilute TNF Receptor (TNF R) in 1×assay medium: Stock is 100 μg/ml For 20 μg/ml: 1:5 dilution: 60 μl of 100 μg/ml of Stock +240 μl of 1×assay medium with wild-type TNF-alpha

Diluted TNF R assay medium containing 20 ng/ml of wild-type TNF-alpha (final on the cell 10 ng/ml) as shown below:

| | Assay medium | Concentration | | Final | |
|---|---|---|---|---|---|
| | (μl) with | Concentration | | in | |
| Stock (μl) | TNF-alpha | dilution plate | | in cells | |
| 300 μl of 20 μg | 300 | 10,000 | ng/ml | 5000 | ng/ml |
| 200 μl of 10,000 ng | 300 | 4000 | ng/ml | 2000 | ng/ml |
| 250 μl of 4000 ng | 250 | 2000 | ng/ml | 1000 | ng/ml |
| 250 μl of 2000 ng | 250 | 1000 | ng/ml | 500 | ng/ml |
| 50 μl of 10,000 μg/ml | 950 | 500 | ng/ml | 250 | ng/ml |
| 200 μl of 500 ng/ml | 300 | 200 | ng/ml | 100 | ng/ml |
| 100 μl of 500 ng/ml | 400 | 100 | ng/ml | 50 | ng/ml |
| 100 μl of 500 ng/ml | 900 | 50 | ng/ml | 25 | ng/ml |
| 200 μl of 50 ng/ml | 300 | 20 | ng/ml | 10 | ng/ml |
| 100 μl 50 ng/ml | 400 | 10 | ng/ml | 5 | ng/ml |
| 50 μl 50 ng/ml | 450 | 5 | ng/ml | 2.5 | ng/ml |
| 0 | 250 | 0 | | 0 | |

All of the above dilutions were done 16 hours prior to adding to the cells. Then 120 μl of each diluted sample was incubated at 4° C., and 120 μl of each sample was incubated at 37° C. The next morning, 50 μl of each sample was added to the cells. The cells were incubated at 37° C. for 4 hours. After 4 hours of incubation, 100 μl of the caspase substrate was added to each well, followed by a 2 hour incubation at 37° C. Read fluorescence. Results are shown in FIG. 6.

TNF-alpha Antagonist Activity of Combinatorial TNF-alpha Variants

Materials and Methods:

Plated cells for the assay: WEHIL64-13Var cells plated at 7.5×105 cells/ml (50 μl/well), incubate at 37degrees C. overnight and prepared Assay Media: (1×, final concentration on cells will be 10 ng/mL) (7 ml full RPMI, 5 uL of 310 ug/mL wild-type his-TNF [Lot#263-56], 140 uL 1 mg/mL ActinomycinD). Dilution of TNF-alpha variants was done as shown below by mixing these samples three days prior to start of experiment:

| | | | Conc. | | | Final | |
|---|---|---|---|---|---|---|---|
| | | | Before | Conc. | | Conc. on | |
| | Stock (uL) | RPMI | 10× | After 10× | | cells | |
| 1 | E146K/N34V/V91E (lot 388-3) 1800 ug/mL: 38.6 | 961.4 | 69,520 | 63,200 | ng/mL | 31,600 | ng/mL |
| | Y115Q/I97T (380-32) 2000 ug/mL: 34.7 | 965.3 | | | | | |
| | Y115Q/I97R (380-32) 1400 ug/mL: 49.8 | 950.2 | | | | | |
| | Y115Q/Y87R (380-32) 1100 ug/mL: 63.3 | 936.7 | | | | | |
| | Y115Q/L57Y (380-32) 1100 ug/mL 63.3 | 936.7 | | | | | |
| | Y115Q/L57F (380-32) 1200 ug/mL 57.8 | 942.2 | | | | | |
| | A145R/L57F (388-3) 2000 ug/mL 34.7 | 965.3 | | | | | |
| | A145R/Y87H (378-96) 880 ug/mL 78.7 | 921.3 | | | | | |
| | Enbrel 25000 ug/mL | 997.3 | | | | | |
| | Buffer (PBS pH 8) 100 uL | 900 | | | | | |
| | TNF R (500 ug/mL) 70 uL | 430 | | | | | |
| 2 | 316 (158 for TNF R) ul of 63,200 ng/mL | 684 | 22,000 | 20,000 | ng/mL | 10,000 | ng/mL |
| | | (342) | | | | | |
| 3 | 316 (158 for TNF R) ul of 20000 ng/mL | 684 | 6,952 | 6,320 | ng/mL | 3,160 | ng/mL |
| | | (342) | | | | | |
| 4 | 316 (158 for TNF R) ul of 6,320 ng/mL | 684 | 2200 | 2,000 | ng/mL | 1000 | ng/mL |
| | | (342) | | | | | |
| 5 | 316 (158 for TNF R) ul of 2000 ng/mL | 684 | 695.2 | 362 | ng/mL | 316 | ng/mL |
| | | (342) | | | | | |
| 6 | 316 (158 for TNF R) ul of 362 ng/mL | 684 | 220 | 200 | ng/mL | 100 | ng/mL |
| | | (342) | | | | | |
| 7 | 316 (158 for TNF R) ul of 200 ng/mL | 684 | 69.52 | 63.2 | ng/mL | 31.6 | ng/mL |
| | | (342) | | | | | |
| 8 | 316 (158 for TNF R) ul of 63.2 ng/mL | 684 | 22 | 20 | ng/mL | 10 | ng/mL |
| | | (342) | | | | | |
| 9 | 316 (158 for TNF R) ul of 20 ng/mL | 684 | 6.95 | 6.32 | ng/mL | 3.16 | ng/mL |
| | | (342) | | | | | |
| 10 | 316 (158 for TNF R) ul of 6.32 ng/mL | 684 | 2.2 | 2 | ng/mL | 1 | ng/mL |
| | | (342) | | | | | |
| 11 | 316 (158 for TNF R) ul of 2 ng/mL | 684 | 0.6952 | 0.632 | ng/mL | 0.316 | ng/mL |
| | | (342) | | | | | |
| 12 | 0 | 684 | 0 | 0 | ng/mL | 0 | |
| | | (342) | | | | | |

After all dilutions were done at 68.4 (34.2 for TNF R) uL of 10×assay media containing WT his TNFa to each dilution well. Then the 96 well was placed in the incubator for 3 days. 50 ul of each sample were added to WEHI164-13Var cells for 4 hours. Upon completion of the incubation, add 100 ul of caspase substrate. Incubate for 1.5 hours. A R110 curve was also prepared by diluting the R110 standard 1:100 in RPMI followed by an 8-point half dilution. Then 100 ul of each dilution were added to a plate without cells, these dilutions are done right before adding the substrate to the cells. 100 ul of subtrate was also added to R110 curve dilutions. Upon the completion of 1.5-hour incubation at 37 degrees C., all samples were read using the Wallac fluoremeter at 484/535 nm wavelengths. Results shown in FIG. 6.

Fixed Equilibrium Screening of TNF-alpha Variants

Prepared 1:10 fixed equilibrium ratios of TNF-alpha variants by mixing together 0.01 mg/mL wild-type his-TNF [lot#263-56] with 0.1 mg/mL variant TNF-alpha in 50 uL reactions in phosphate-buffered saline (PBS).

| | | | Volume | 0.33 mg/ | |
|---|---|---|---|---|---|
| | | Conc. | Prot. | mL wt | |
| Protein Name | Lot# | (mg/mL) | (uL) | TNF (uL) | PBS |
| Y115Q/L57W | 380-32 | 1.3 | 3.85 | 1.5 | 44.65 |
| Y115M/D143N | 380-32 | 0.36 | 13.8 | 1.5 | 34.7 |
| Y115Q/Y87H | 380-32 | 1.1 | 4.55 | 1.5 | 44 |
| Y115Q/A145R | 380-32 | 0.53 | 9.4 | 1.5 | 39.1 |
| Y115Q/A145F | 380-32 | 2.0 | 2.5 | 1.5 | 46 |
| Y115Q/L57Y | 380-32 | 1.1 | 4.55 | 1.5 | 44 |
| Y115M/A145R | 380-32 | 0.74 | 6.8 | 1.5 | 41.7 |
| Y115M/E146K | 380-32 | 0.27 | 18.5 | 1.5 | 30 |
| Y115M/D143Q | 380-32 | 0.37 | 13.5 | 1.5 | 35 |
| Y115Q/L57F | 380-32 | 1.2 | 4.17 | 1.5 | 44.3 |
| A145R/I97R | 380-32 | 0.56 | 9 | 1.5 | 39.5 |
| A145R/Y87H | 380-32 | 1.6 | 3.13 | 1.5 | 45.4 |
| A145R/L75Q | 380-32 | 0.86 | 5.8 | 1.5 | 42.7 |
| A145R/L75K | 380-32 | 0.99 | 4.9 | 1.5 | 43.6 |
| Y115M/A145R | 380-32 | 0.23 | 21.7 | 1.5 | 27 |
| A145R/S86Q | 380-32 | 1.2 | 4.2 | 1.5 | 44.3 |
| E146K/V91E/N34E | 380-32 | 1.2 | 2.8 | 1.5 | 45.7 |
| A145R/S86R | 378-95 | 0.27 | 18.5 | 1.5 | 30 |
| A145R/I97T | 378-97 | 0.47 | 10.6 | 1.5 | 37.9 |
| A145R/L75E | 378-94 | 1.73 | 2.9 | 1.5 | 45.6 |
| Y115Q/S86R | 380-32 | 0.94 | 4.9 | 1.5 | 43.6 |
| Y115Q/Y87R | 380-32 | 1.1 | 4.6 | 1.5 | 43.9 |
| Y115Q/L75K | 380-32 | 0.75 | 6.7 | 1.5 | 41.8 |
| Y115Q/S86Q | 380-32 | 1.0 | 4.9 | 1.5 | 43.6 |
| Y115Q/E146K | 380-32 | 0.38 | 13.1 | 1.5 | 35.4 |
| Y115Q/L75Q | 380-32 | 0.58 | 8.6 | 1.5 | 39.9 |
| Y115Q/I97T | 380-32 | 2.0 | 2.5 | 1.5 | 46 |
| Y115Q/D143N | 380-32 | 0.3 | 16.7 | 1.5 | 31.8 |
| Y115Q/L75E | 380-32 | 0.62 | 8.1 | 1.5 | 40.4 |
| Y115Q/I97R | 380-32 | 1.4 | 3.6 | 1.5 | 44.9 |
| A145R/L57F | 388-3 | 2 | 2.5 | 1.5 | 46 |

Prepared this mixture and incubated at 37 degrees C. for 3-4 days. Plated cells for the assay: Human U937 cells plated at 1×106 cells/ml (50 μl/well) and incubated at 37 degrees C. overnight.

Caspase Assay

Warmed full RPMI medium and supplement with 2 ug/mL Actinomycin D. Mixed each entire 50 uL reaction with 450 uL Actinomycin D supplemented RPMI medium. This mixture was diluted 1:1 eleven times to generate a dose curve for the fixed equilibrium. 50 uL of the dilution mixture was applied to the cells in quadruplicate. Cells were incubated in the TNF-alpha/TNF-alpha variant fixed equilibrium for 1.5 hours. Upon completion of the incubation, 100 ul of caspase substrate was added and then incubated for 1.5 hours. A R110 curve was also prepared by diluting the R110 standard 1:100 in RPMI followed by an 8-point half dilution. Then 100 ul of each dilution was added to a plate without cells, these dilutions are done right before adding the substrate to the cells. 100 ul of substrate was also added to R110 curve dilutions. Upon the completion of 1.5-hour incubation at 37 degrees C., all samples were read using the Wallac fluoremeter at 484/535 nm wavelengths. Results are shown in FIGS. 6A-C.

Binding Assay

Biotinylation of TNFa was performed by adding 20 molar excess Sulfo-NHS-LC-biotin to the protein sample and incubating the sample on ice for 2 hours. Excess biotin was removed from the sample by dialysis. Coupling ratios ranged between 1 to 4. The protein concentration of biotinylated TNFa was determined by BCA protein assay (Pierce). Wells of a microtiter plate were coated with anti-FLAG antibody at a concentration of 2.5 mg/ml and blocked with 3% BSA overnight at 4° C. The FLAG-tagged protein TNFR1 receptor was added at a concentration of 10 ng/ml in PBS +1% BSA to wells of the anti-FLAG-coated microtiter plate, and the plate was incubated for 2 hours at room temperature. Biotinylated TNFa proteins ranging in concentrations from 0-1 mg/mL were added in quadruplicate to anti-FLAG-TNFR1-coated wells to represent total binding. Non-specific binding was measured by adding biotinylated TNF-alpha proteins ranging in concentrations from 0-1 μg/ml in quadruplicate to wells coated only with anti-FLAG antibody. Binding was allowed to occur overnight at +4° C. to ensure equilibrium. Alkaline phosphatase conjugated neutravidin (Pierce) was added to the wells at 1:10,000 dilution in PBS +1% BSA and incubated for 30 min at room temperature. Luminescence was detected upon the addition of the CSPD star substrate (Applied Biosystems, Foster City, Calif.) and was measured (Wallac VICTOR, Perkin Elmer Life Sciences, Boston, Mass.). The specific binding of TNFa was calculated by subtracting non-specific binding from total binding. Data was fit to the binding equation y=(BLmax*x)/(Kd+x).

The results of the binding assays are shown in FIG. 6. All variants show a decrease in receptor binding.

### Example 7

TNF-alpha Variants Exchange with Wild-type TNF-alpha to Reduce Activation of NFk B

TNF-alpha variants tested were A145R, double variant A145R/Y87H, and triple variant E146K/V91E/N34E. His-tagged TNF-alpha was pre-incubated with 10-fold excess (1:10) of different variants for 3-days at 37 degrees C. Wild-type TNF-alpha alone and pre-exchanged heterotrimers of TNF-alpha variants were then tested for their ability to activate an NFkB-driven luciferase reporter (pNFkB-luc, Clontech) in 293T cells. 293T cells were seeded at 1.2×104 cells/well in 96-well plates. Cells were then transfected with pNFkB-luc (NF-kB dependent luciferase reporter) or pTal (Control: basal promoter driving the luciferase gene, but without NFkB binding elements) using Fugene transfection reagent according to the manufacturer's protocol (Roche). 12 hrs after transfection, cells were treated with a final concentration of 10 ng/ml wild-type TNF-alpha or a pre-exchanged mixtures of 10 ng/ml: TNF/100 ng/ml variant. 12 hrs after treatment, the cells in 96-well plates were processed for the luciferase assay using the Steady-Glo Luciferase Assay System (Promega) according to the manufacturer's protocol. Luminescence from each well was measured using the Packard TopCount NXT (Packard Bioscience) luminescence counter. Treated samples were tested in quadruplicates, and mean values of luminescence were plotted as bar values including the standard deviation for each treatment. The results are shown in FIG. 20A. The graph shows that the TNF-alpha variants of the present invention were effective in decreasing wild-type TNF-alpha induced NFkB activation. The TNF-alpha variant A145R/Y87H was most effective in decreasing TNF-alpha induced NFkB activation.

Immuno-localization of NFkB in HeLa Cells

HeLa cells were seeded onto 12 mm sterile coverslips (Fisherbrand) at a density of 1.5×105 cells/well in 6-well plates and cultured at 37 degrees C. at 5% CO2 atmosphere. The following day, the cells were treated with various concentrations of his-tagged wild-type TNF-alpha, A145R/Y87H variant alone, or the combination of the his-tagged TNF-alpha and 10-fold excess of the A145/Y87H variant (pre-exchanged for three days at 37 C.) at 37° C., 5% CO2. After 30 minutes of incubation, the cells attached to coverslips in 6-well plates were briefly washed with PBS and fixed in 4% formaldehyde/PBS for 10 minutes. Cells were then washed an additional five times with PBS or maintained in the last PBS wash overnight before processing cells for immunocytochemistry. Fixed cells on coverslips were then treated with 0.1% Triton X-100/PBS. The buffer was aspirated and cells on coverslips were blocked in a humidified chamberfor 15 minutes with 50 ul of 0.1% BSA/0.1% TX-100/PBS per coverslip at 37° C. The blocking reagent was then removed and replaced with primary antibody against p65 subunit of NF-kB (pAb C-20, Santa Cruz Bioscience). After one hour of incubation at 37 degrees C., the antibody was removed and coverslips were washed 5 times with PBS. 50 ul of FITC-conjugated secondary antibody (Jackson Immuno laboratories) diluted in blocking buffer (1:100) was added to each coverslip (Jackson Immunolaboratories) and coverslips were incubated in a light-safe humidified chamber for an additional hour before removing the secondary antibody with 5 washes of PBS. Coverslips were briefly rinsed with d-water, air-dried in a light-safe chamber and mounted onto slides using Anti-fade (Molecular Probes). Digital images of antibody-reacted cells were captured using a FITC filter and 40×objective on a Nikon Eclipse TS100 microscope coupled to a Cool SNAP-Pro CCD camera (Media Cybernetics) and operated using Image Pro Plus software (Media Cybernetics).

FIG. 4B shows photographs of the immuno-localization of NFkB in HeLa cells showing that the exchange of wild-type TNF-alpha with the A145/Y87H TNF-alpha variant inhibits TNF-alpha-induced nuclear translocation of NFkB in HeLa cells. The TNF-alpha variant A145R/Y87H alone does not induce NFkB nuclear translocation, unlike the wild-type TNF-alpha. Moreover, the wild-type TNF-alpha exchanged (3-days, 37 degrees C.) to form heterotrimers with excess variant (10 fold excess of TNF-alpha variant A145R/Y87H) loses its ability to induce NFkB nuclear translocation. This data is consistent with the effects of this variant in the luciferase reporter assay.

TNFA Variant A145R/Y87H Reduced TNF-alpha Induced Activation of the NFkB-driven Luciferase Reporter

His-tagged wild-type TNF-alpha, TNF-alpha variant A145/Y87H and the exchanged wild-type TNF-alpha:A145R/Y87H heterotrimer (1-day exchange with 10-fold excess TNF-alpha variant A145R/Y87H at 37 degrees C.) were tested in the NFkB luciferase reporter assay as in Example 7A above. The experiment was carried out as in Example 7A, with the exception that a wider range of final TNF-alpha concentrations and increasing doses were used (0.78, 1.56, 3.13, 6.25,12.5, 25 ng/ml) with 10-fold excess of TNF-alpha variant (A145R/Y87H) at each TNF-alpha concentration.

The wild-type TNF-alpha:A145R/Y87H heterotrimer has a significantly reduced activation level, indicating the TNF-alpha A145R/Y87H variant's inhibitory effect on wild-type TNF-alpha. Unlike wild-type TNF-alpha, the TNF-alpha variant A145/Y87H alone has no significant agonizing effect on NFkB activation as shown by the lower dotted line in FIG. 4C. Wild-type TNF-alpha induced activation is dependent on the NFkB activation as the reporter and without NFkB binding elements is unresponsive to the TNF-alpha as shown in the solid gray line in FIG. 4C.

Antagonistic RANKL Variant Library Design

Antagonistic RANKL variants were generated using the dominant-negative strategy which includes the single chain variety, and by the selection of competitive inhibitor antagonists.

Dominant-Negative RANKL Library Variants

PDA™ was utilized to generate a human structural model of RANKL in addition to selecting amino acids that disrupt RANK binding. The lack of a crystal structure for human OPGL necessitated the creation of a homology model where PDATM was used for side chain placement based on the human sequence and mouse RANKL structure (PDB#1JTZ). This modeling was facilitated by the 87% sequence identity between the mouse and human RANKL sequences. The dominant-negative RANKL therapeutic strategy is based on the design of novel RANKL variants that have reduced receptor binding and/or activation properties and the ability to heterotrimerize with wild-type RANKL (FIG. 21). In other words, RANKL variants that do not activate RANK will exchange with wild-type RANKL protein and sequester it into inactive heterotrimers, inhibiting its activity. The number and activity of osteoclasts will be lowered as a result of this treatment leading to a decrease in bone resorption and an overall increase in bone mineral density.

The dominant-negative RANKL variants were designed by substituting the amino acids at key RANKL-RANK contact points with amino acids that disrupt the ability of the ligand to activate receptor. PDATM technology was used to select appropriate substitutions for optimal protein folding. The exchange of these trimeric RANKL variants with trimeric wild-type RANKL will result in the deactivation of the wild-type RANKL and reduced osteoclast formation. To help accomplish this goal more effectively, the RANKL variants can also be designed to preferentially heterotrimerize with wild-type RANKL.

Single Chain Dominant-Negative Polypeptides

Multiple strategies for covalent linkage of monomers exist. These included, but are not limited to: polypeptide linkages between N and C-termini of two domains, made up of zero or more amino acids (resulting in single chain polypeptides comprising multiple domains); linkage via a disulfide bond between monomers; linkage via chemical crosslinking agents.

Multiple strategies exist for modification of individual domains such that receptor binding is removed (or reduced). These include, but are not limited to: amino acid modifications that create steric repulsion between ligand domain and receptor; modifications that create electrostatic repulsion; modifications that create unfavorable desolvation of amino acids; and chemical modification of amino acids at the ligand/receptor interface (e.g. PEGylation or glycosylation).

Linkage of RANKL Monomers into a Trimer

To improve the dominant negative behavior of these mutants, single-chain dimers between two modified receptor interaction domains are being created.

RANKL Variant Library Construction

The full-length RANKL cDNA was cloned using RT-PCR on lymph node cDNAs. Two lengths of RANKL, amino acids 147 to 317 and 159 to 317, were subcloned into pET33b after PCR-mediated addition of TEV and HIS tags.

Muatagenesis reactions were performed to introduce the nucleotide changes that result in the desired amino acid substitutions. The mutagenesis reactions utilize pairs of divergent and overlapping oligonucleotides with nucleotide variations that introduce the specific amino acid changes. These oligonucleotides are extended using Pfu polymerase through multiple rounds of thermal cycling and these extension products are transformed into TOP10 bacterial cells after digesting the products with Dpnl. Clones were sequenced to confirm the designed changes and to insure no modifications were introduced into the cDNA during the thermal cycling.

RANKL Variant Screening for Non-agonists, Superagonists, and Antagonists

A library of 89 human RANKL variants was constructed with all members of the library comprising the amino acid sequence in FIG. 1b and the two solubility-imparting modifications, C221S/1247E in addition to the 89 computationally designed inhibitory modifications. These 89 human RANKL variant proteins were expressed and purified from E. coli and 52 were screened for non-agonists, superagonists, and antagonists in an assay that monitors osteoclastogenesis in RAW264.7 cells. Treating RAW264.7 cells with the purified human RANKL variant protein and measuring the amount of TRAP released identified non-agonist and superagonist variants. Of the 52 RANKL variants screened, 21 RANKL variants were identified to be non-agonists, while C221S/1247E/A172R was found to be a superagonist. In addition, the antagonism screens described above identified the six antagonizing variants. These antagonizing variants have the ability to lower the osteoclastogenesis activity of human RANKL.

Tartrate-Resistant Acid Phosphatase (TRAP) Activity Bioassay

The TRAP activity bioassay was used for assaying non-agonism, super-agonism, and antagonism. TRAP activity was measured after adding the RANKL variant protein to cell lines such as RAW264.7 to identify RANKL variants that are non-agonists or super-agonists (FIGS. 23-25, 29). An additional screen was performed to identify antagonist RANKL variants by mixing RANKL variant protein with one of the active RANKL solubility variants, C221S/1247E, and adding the protein mixture to the RAW264.7 and measuring TRAP activity. Antagonist RANKL variants were identified that lowered the activity of the RANKL solubility variant C221S/1247E.

RAW 264.7 cells (ATCC#TIB-71) were cultured and maintained in 75 cm2 flasks in 5% CO2 humidified incubator at 37° C. in growth media contairing a-MEM w/2 mM L-Glutamine (Gibco-BRL#12571-063), 10% Heat Inactivated Fetal Bovine Serum (FBS) (Hyclone#SH30071.03) and Penicillin/Streptomycin (Gibco-BRL#15140-122) 100 units/ml and 100 ug/ml respectively.

Recombinant mouse RANKL (R&D#462-TR), recombinant human soluble RANKL (Biosource#PHP0034), and recombinant human soluble RANKL variants were diluted to their working concentration in assay media consisting of a-MEM w/ I-glutamine (Gibco-BRL#12571-063) supplemented with 10% Heat Inactivated Fetal Bovine Serum (FBS) (Hyclone#SH30071.03) and Penicillin/Streptomycin (Gibco-BRL#15140-122) 100 units/ml and 100 ug/ml respectively. 250 ml of RANKL standards and samples were added to 96-well assay plate in triplicate or quadruplicate, and serially diluted 1:2 by transferring 125 ml into subsequent wells containing 125 ml of assay media.

Aspirating the exhausted media, adding 10 ml of pre-warmed media to the flask, and scraping cells from the flask using a cell scraper harvested the cells. The cells were transferred to a 15 ml conical tube, spun at 1000 rpm for 5 minutes and resuspended in 10 ml of assay media. The cells were counted and seeded at density of 1.5×10³/125 ml/well in 96-well assay plate. Assay plates were incubated at 37° C. in 5% CO2 humidified incubator for 72 hours.

The amount of osteoclast-like cells induced from the RAW 264.7 cells was determined by measuring the Tartrate-Resistant Acid Phosphatase (TRAP) activity. TRAP substrate was prepared by adding 20 mg of p-nitrophenol phosphate (pNPP) (ICN#100878) to 10 ml TRAP buffer pH5.0 (100 mM Sodium Acetate, 11.5 mg/ml Sodium Tartrate (ICN#195503)). The media was aspirated off the assay plate and discarded. Cells were fixed for 1 minute with 1:1 ethanol and acetone solution and washed once with 100-150 ml/well of PBS (Hyclone#SH30256.02). 100 ml of TRAP solution was added to each well, and the plate was incubated at 37° C. for 2 hours. The reaction was stopped by adding 50 ml/well of a 0.2 N NaOH solution and the absorbance was read at 405 nm. If the absorbance reached saturation, the solutions were diluted 1:5 and read again.

Tartrate-Resistant Acid Phosphatase (TRAP) Cytological Staining

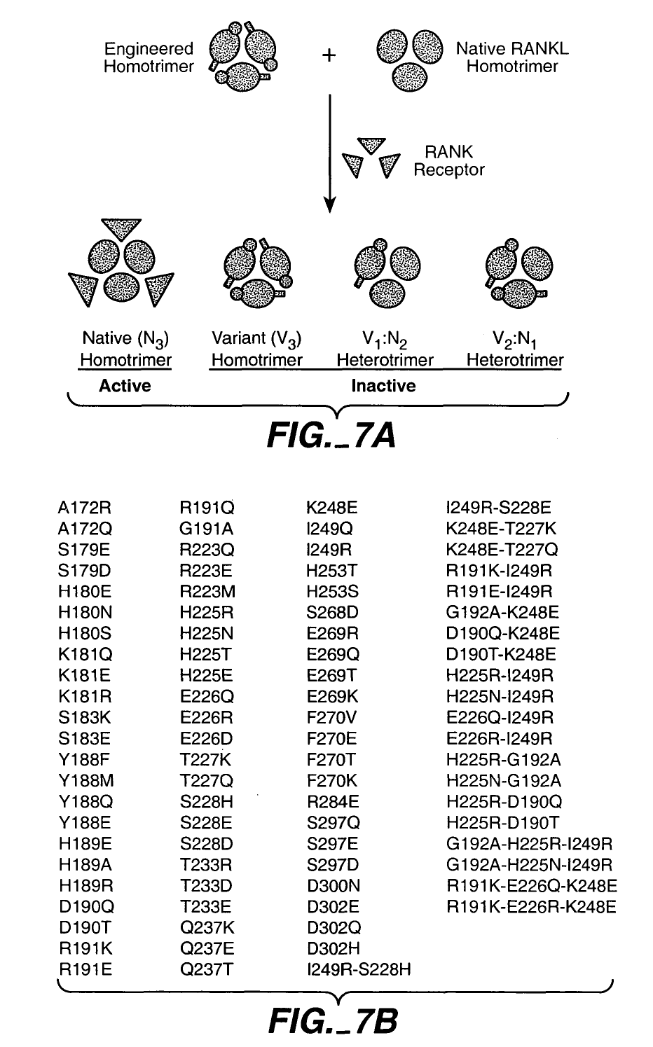

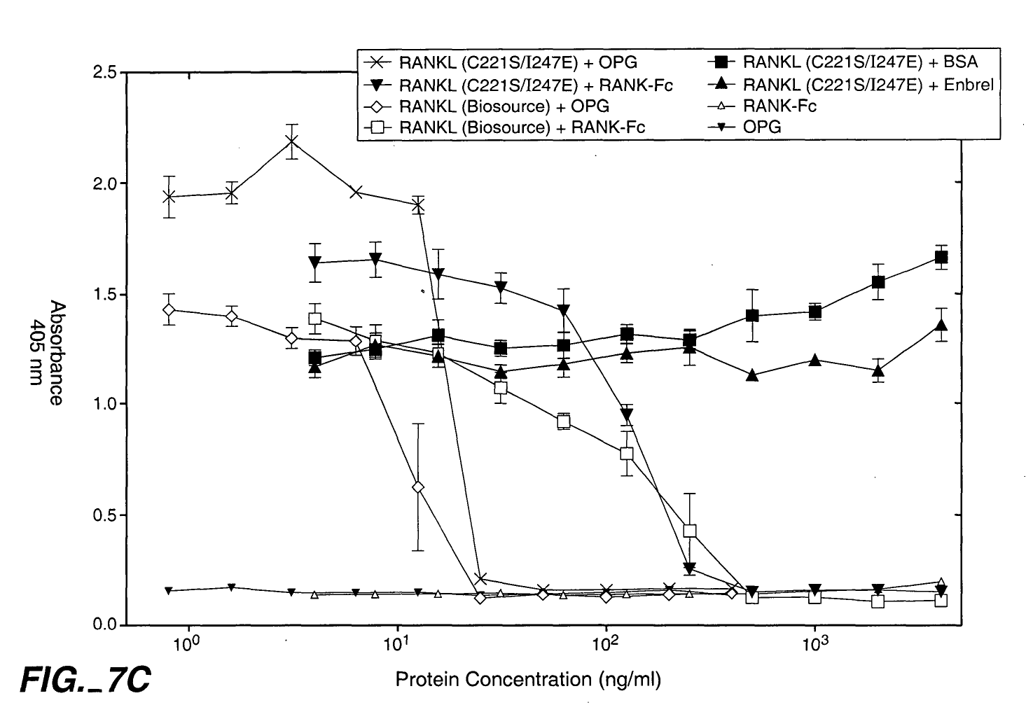

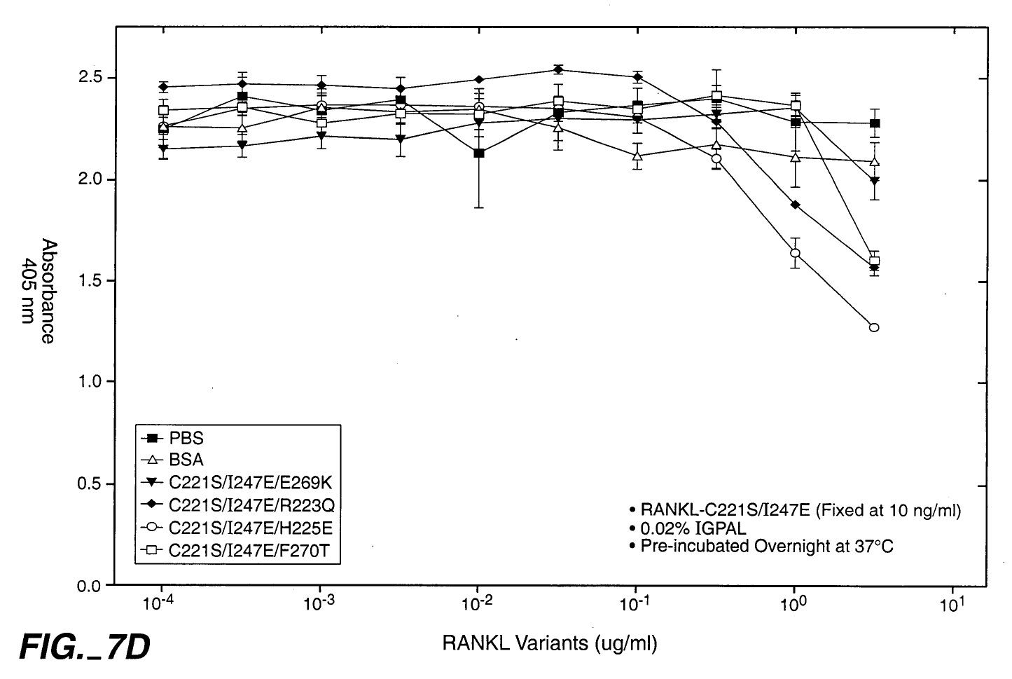

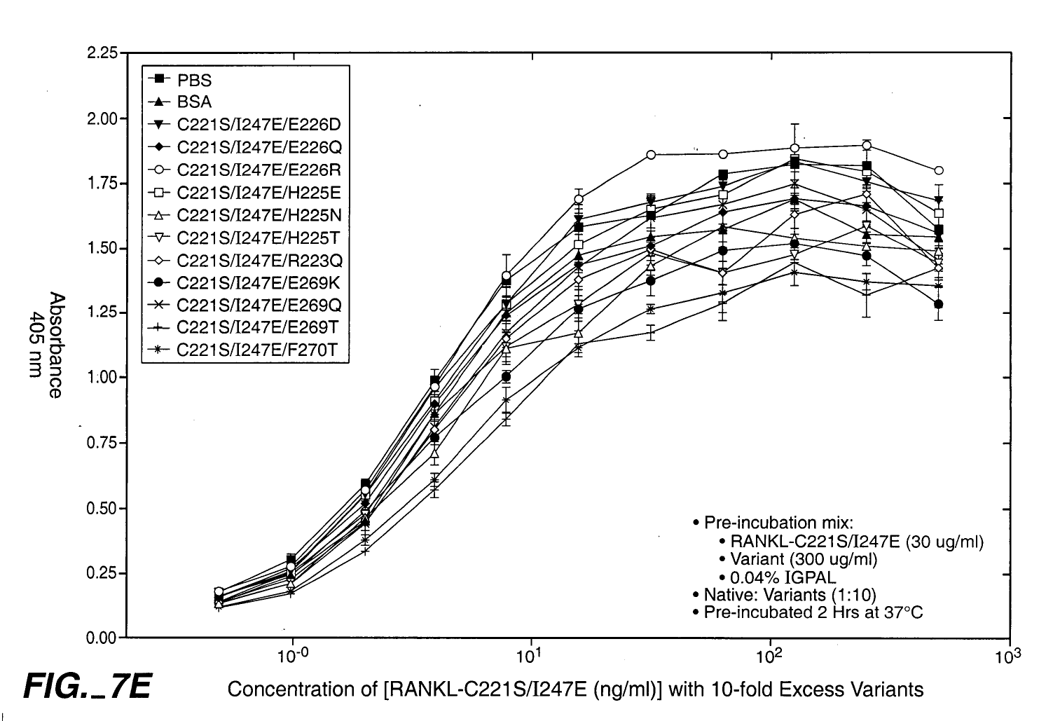

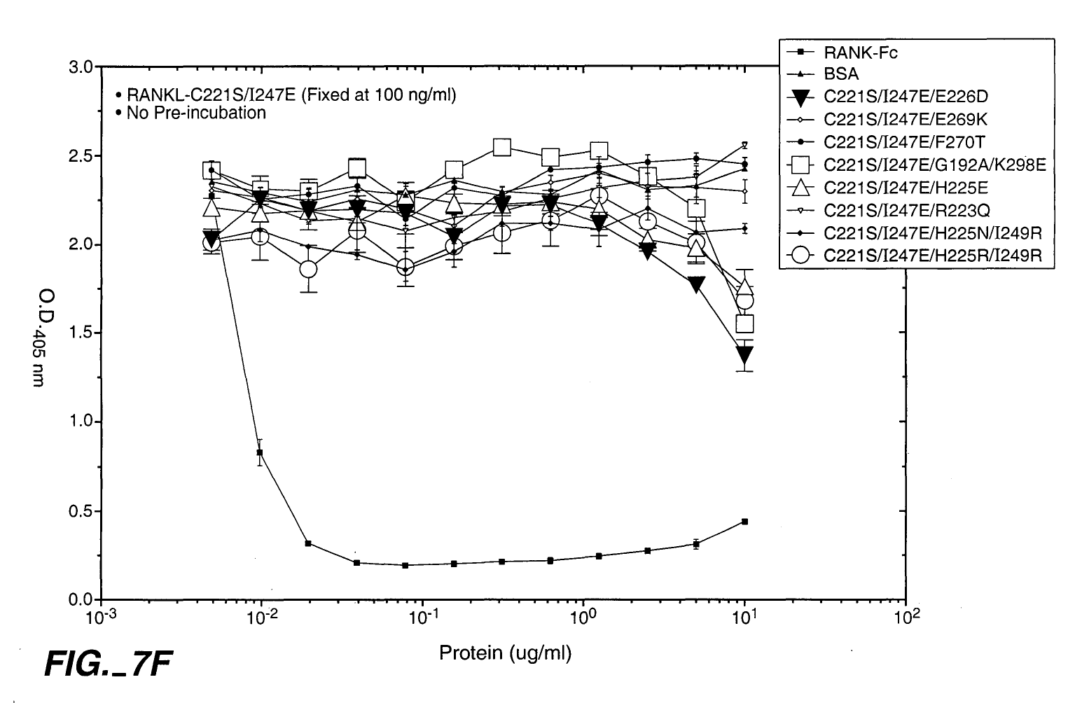

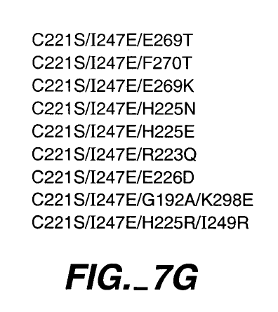

In parallel experiments, osteoclast formation from the RAW 264.7 cells was measured by the presence of Tartrate-Resistant Acid Phosphatase (TRAP) multinucleated positive cells using cytological staining. 0.1M Acetate buffer was prepared prior to staining by combining 35.2 ml of a 0.2M Sodium Acetate solution, 14.8 ml of a 0.2M Acetic Acid solution and 50 ml of water. 10 ml TRAP buffer pH5.0 (50 mM Acetate Buffer, 30 mM Sodium Tartrate (Sigma#S-8640), 0.1 mg/ml Napthol AS-MX Phosphate Disodium Salt (Sigma#N-5000), 0.1% Triton X-100) was prepared fresh for each assay. The TRAP buffer was warmed in a 37° C. water bath and 0.3 mg/ml of Fast Red Violet LB Stain (Sigma#F-3381) was added and the stain was returned to the 37° C. water bath. The media was aspirated off the assay plate and discarded. The cells were washed once with 150 ml/well of PBS (Hyclone#SH30256.02) and subsequently fixed with 100 ml/well of a 10% Glutaraldehyde solution for 15 minutes at 37° C. The cells were washed twice with 150 ml/well of pre-warmed PBS. 100 ml of the pre-warmed TRAP stain was added to each well and the plate was incubated at 37° C. for 5-10 minutes. The TRAP stain was removed and 100 ml of PBS was added to each well to prevent cells from drying out. TRAP positive multinucleated cells (more than three nuclei) were counted. FIG. 7 shows the results.

Other TNFSF Variants

BlyS, CD40L, APRIL, OX-40 are generated according to the protocol disclosed above. They have exchange with their corresponding wild-type TNFSF member. APRIL and BlyS have exchange between them and the corresponding APRIL or BlyS. Binding assays, and dose response curves are done for each TNFSF member.

Whereas particular embodiments of the invention have been described above for purposes of illustration, it will be appreciated by those skilled in the art that numerous variations of the details may be made without departing from the invention as described in the appended claims.

We claim:

1. A method of antagonizing a naturally occurring TNFSF protein comprising contacting a naturally occurring TNFSF monomer protein with a variant TNFSF monomer protein comprising at least a variant extracellular domain of a TNFSF protein, to form a mixed TNFSF oligomer.

2. A method according to claim 1 wherein said mixed oligomers have reduced receptor signaling as compared to wild-type oligomers.

3. A method according to claim 2 wherein said mixed oligomers are substantially incapable of activating receptor signaling.

4. A method according to claim 1 wherein said mixed oligomer interacts with a receptor interface in at least one receptor binding site to render said receptor substantially incapable of activating receptor signaling.

5. A method according to claim 1 wherein said variant TNFSF monomer protein comprises at least one receptor contact domain that has reduced affinity for a desired receptor as compared to its corresponding wild-type TNFSF protein and retains the ability to interact with other TNFSF monomers.

6. A method according to claim 1, wherein said variant TNFSF monomer protein physically interacts with a naturally occurring TNFSF monomer protein to reduce the ability of the naturally occurring TNFSF to activate at least one receptor.

7. A method according to claim 1, wherein said variant TNFSF protein is a fusion protein.

8. A method according to claim 1 wherein said variant protein is chemically modified.

9. A method according to claim 8, wherein said chemical modification is PEGylation.

10. A method according to claim 8, wherein said mixed oligomer comprises covalent cross-linkages between monomers.

11. A method according to claim 10, wherein said mixed oligomer comprises at least one linker peptide between monomers.

12. A method according to claim 11, wherein said linker peptide is a sequence of at least one and not more than about 30 amino acid residues.

13. A method according to claim 12, wherein said linker peptide is a sequence of at least 5 and not more than about 20 amino acid residues.

14. A method according to claim 13, wherein said linker peptide is a sequence of at least 10 and not more than about 15 amino acid residues.

15. A method according to claim 11, wherein the linker peptide comprises at least one of the following amino acid residues: Gly, Ser, Ala, or Thr.

16. A method of making a mixed TNFSF oligomer comprising contacting at least one variant TNFSF protein comprising at least a variant extracellular domain of a TNFSF monomer protein with a homo-oligomer comprising naturally occurring TNFSF monomer proteins, under conditions whereby at least one naturally occurring TNFSF monomer exchanges with a variant monomer to form a mixed oligomer.

17. A mixed TNFSF oligomer comprising at least one variant TNFSF protein comprising at least a variant extracellular domain of a TNFSF monomer protein and a naturally occurring TNFSF monomer protein.

18. A variant TNFSF monomer protein comprising at least a variant extracellular domain of a TNFSF protein.

19. A variant TNFSF protein wherein said variant TNFSF protein will interact with a receptor interface in at least one receptor binding site to render said receptor substantially incapable of activating receptor signaling.

20. A variant TNFSF protein according to claim 18 comprising at least one receptor contact domain that has reduced affinity for a desired receptor as compared to its corresponding wild-type TNFSF protein and retains the ability to interact with other receptor interaction domains.

21. A mixed TNFSF oligomer comprising at least one variant TNFSF protein monomer according to claim 18, wherein said mixed oligomer has reduced ability to activate the corresponding receptor as compared to a wild-type oligomer.

22. A variant TNFSF protein according to claim 18, wherein said variant TNFSF protein physically interacts with a naturally occurring TNFSF protein to form mixed trimers.

23. A mixed TNFSF oligomer comprising at least one variant TNFSF protein monomer according to claim 18 comprising a modification at a receptor contact position.

24. A variant TNFSF monomer protein according to claim 18, wherein said variant TNFSF protein comprises a modification at a trimer interface position.

25. A variant TNFSF protein according to claim 18, wherein said variant TNFSF protein physically interacts with its corresponding naturally occurring TNFSF protein.

26. A variant TNFSF protein according to claim 18, wherein said variant TNFSF protein physically interacts with a non-corresponding naturally occurring TNFSF protein.

27. A recombinant nucleic acid encoding the variant TNFSF monomer protein of claims 18-26.

28. An expression vector comprising the recombinant nucleic acid of claim 27.

29. A host cell comprising the recombinant nucleic acid of claim 27.

30. A host cell comprising the expression vector of claim 28.

31. A method of producing a non-naturally occurring TNFSF protein comprising culturing the host cell of claim 29 or 30 under conditions suitable for expression of said nucleic acid.

32. A method according to claim 31, further comprising recovering said TNFSF protein.

33. A pharmaceutical composition comprising a variant TNFSF protein according to claims 18-26 and a pharmaceutically acceptable carrier.

34. A method for treating a TNFSF related disorder comprising administering a variant TNFSF protein of claim 18-26 to a patient in need of said treatment.

35. A method according to claim 34, wherein said TNFSF related disorder is an autoimmune disease.

36. A variant TNFSF protein according to claims 18, wherein at least one modification is non-conservative.

37. A variant TNFSF protein according to claims 18, wherein at least one modification is a surface modification.

38. A variant TNFSF protein according to claims 36, wherein said modification is located within a domain selected from the group consisting of: Large Domain, Small Domain, DE Loop, Trimer Interface and combinations thereof.

39. A variant TNFSF protein according to claim 38, wherein at least one of said Large Domain positions is selected from the group consisting of TNFA corresponding positions 28, 29, 30, 31, 32, 33, 34, 63, 64, 65, 66, 77, 68, 69, 112, 113, 114, 115, 137, 138, 139, 140, 141, 142, 143, 144, 145, 146 and 147.

40. A variant TNFSF protein according to claim 38, wherein at least one of said Small Domain positions is selected from the group consisting of TNFA corresponding positions 72, 73, 74, 75, 76, 78, 79, 95, 96, 97 and 98.

41. A variant TNFSF protein according to claim 38, wherein at least one of said DE Loop positions is selected from the group consisting of TNFA corresponding positions 84, 85, 86, 87, 88 and 89.

42. A variant TNFSF protein according to claim 38, wherein at least one of said Trimer interface positions is selected from the group consisting of TNFA corresponding positions 11, 13, 15, 34, 36, 53, 54, 55, 57, 59, 61, 63, 72, 73, 75, 77, 119, 87, 91, 92, 93, 94, 95, 96, 97, 98, 99, 102, 103, 104, 109, 112, 113, 114, 115, 116, 117, 118, 119, 120, 121, 122, 123, 124, 125, 147, 148, 149, 151, 155, 156 and 157.

43. A variant TNFSF protein according to claim 42, wherein at least one of said Trimer interface positions is selected from the group consisting of: 57, 34, and 91.

44. A variant TNFSF protein according to claim 18, wherein said variant TNFSF protein antagonizes soluble naturally occurring TNFSF proteins.


SEQUENCE LISTING (23 sequences)

SEQ ID NO: 1 - protein, 147 aa, Homo sapiens
KPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 2 - protein, 144 aa, Homo sapiens
KPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 3 - protein, 138 aa, Homo sapiens
RKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 4 - protein, 148 aa, Homo sapiens
NPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMV

SEQ ID NO: 5 - protein, 140 aa, Homo sapiens
NQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 6 - protein, 157 aa, Homo sapiens
LPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMV

SEQ ID NO: 7 - protein, 136 aa, Homo sapiens
HSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 8 - protein, 141 aa, Homo sapiens
QDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKL

SEQ ID NO: 9 - protein, 141 aa, Homo sapiens
QIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 10 - protein, 151 aa, Homo sapiens
QPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKV

SEQ ID NO: 11 - protein, 161 aa, Homo sapiens
QRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLV

SEQ ID NO: 12 - protein, 126 aa, Homo sapiens
GPQQDPRLYWQGGPALGRSFLHGPELDKGQLRIHRDGIYMVHIQVTLAICSSTTASRHHPTTLAVGICSPASRSISLLRLSFHQGCTIVSQRLTPLARGDTLCTNLTGTLLPSRNTDETFFGVQWV

SEQ ID NO: 13 - protein, 153 aa, Homo sapiens
RQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRV

SEQ ID NO: 14 - protein, 98 aa, Homo sapiens
GEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQV

SEQ ID NO: 15 - protein, 138 aa, Homo sapiens
APFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLY

SEQ ID NO: 16 - protein, 111 aa, Homo sapiens
KGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 17 - protein, 113 aa, Homo sapiens
CMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILL

SEQ ID NO: 18 - protein, 33 aa, Artificial sequence
  coiled-coil motif
RMEKLEQKVKELLRKNERLEEEVERLKQLVGER

SEQ ID NO: 19 - protein, 24 aa, Artificial sequence
  coiled-coil motif
AALESEVSALESEVASLESEVAAL

SEQ ID NO: 20 - protein, 24 aa, Artificial sequence
  coiled-coil motif
LAAVKSKLSAVKSKLASVKSKLAA

SEQ ID NO: 21 - protein, 5 aa, Artificial sequence
  linker consensus sequence
GSGGS

SEQ ID NO: 22 - protein, 5 aa, Artificial sequence
  linker consensus sequence
GGGGS

SEQ ID NO: 23 - protein, 4 aa, Artificial sequence
  linker consensus sequence
GGGS